(19) United States
(12) Patent Application Publication
(10) Pub. No.: US 2023/0399637 A1
(43) Pub. Date: Dec. 14, 2023

(54) CROSSLINKED POROUS PROTEIN CRYSTALS WITH GUEST BARCODE DNA

(71) Applicant: COLORADO STATE UNIVERSITY RESEARCH FOUNDATION, Fort Collins, CO (US)

(72) Inventors: Christopher D. SNOW, Fort Collins, CO (US); Julius D. STUART, Eaton, CO (US); Rebekah C. KADING, Fort Collins, CO (US); Lyndsey I. GRAY, Fort Collins, CO (US); Daniel A. HARTMAN, Ithaca, NY (US)

(21) Appl. No.: 18/252,143

(22) PCT Filed: Nov. 10, 2021
(86) PCT No.: PCT/US2021/072324

§ 371 Date: May 8, 2023

Related U.S. Application Data
(60) Provisional application No. 63/111,927, filed on Nov. 10, 2020.
(60) Provisional application No. 63/248,764, filed on Sep. 27, 2021.

(51) Int. Cl. C12N 15/10; C12Q 1/686

(52) U.S. Cl. CPC C12N 15/1068; C12Q 1/686

(57) ABSTRACT

The present disclosure relates to data storage systems and methods of making thereof. Aspects of the disclosure further relate to engineered porous protein crystals that bind and adsorb guest information storage mediums such as DNA.

### CROSS REFERENCE TO RELATED APPLICATION

This application claims the benefit of U.S. Provisional Application No. 63/111,927 filed on Nov. 10, 2020, and U.S. Provisional Application No. 63/248,764 filed on Sep. 27, 2021, the disclosures of which are each hereby incorporated by reference in their entirety.

### GOVERNMENTAL RIGHTS

This invention was made with government support under grant R21 AI146740 awarded by the National Institutes of Health and grant 1704901 awarded by the National Science Foundation. The U.S. government has certain rights in the invention.

### STATEMENT REGARDING SEQUENCE LISTING

The instant application contains a Sequence Listing which has been submitted via ASCII copy created on Nov. 10, 2021, referred to as ‘CSURF_065620-707508_SEQ_ST25.txt’ that is 4 kilobytes (KB) in size having 11 sequences and is incorporated herein in its entirety for all purposes.

### FIELD OF THE INVENTION

The present disclosure relates to data storage systems and methods of making thereof. Aspects of the disclosure further relate to engineered porous protein crystals that bind and adsorb guest information storage mediums such as DNA.

### BACKGROUND OF THE INVENTION

Marking materials with a unique symbol, tattoo, or signature is a common technique for purposes such as, but not limited to, monitoring product flow through supply chains, maintaining product inventory, assessing authenticity, and determining product age. Applying a unique marker to a material and/or organism also provides a way of detecting fake, or counterfeit materials such as pharmaceuticals, currency, or munitions. A canonical practice for labeling materials throughout industry is by including a barcode on the product labeling that can be later scanned for downstream monitoring. While a widely used and practical technique, current external, visual labeling of barcodes on products fails to conceal the unique marker, thus allowing the opportunity for subversive duplication that is likely to go undetected, compromising supply chain integrity. Current forms of barcodes that attempt to address some of the criteria for unique markers, such as watermarking currency and product packaging, remain severely limited in the amount of stored information. Accordingly, there is a need for improved marking systems.

A growing field of research is currently exploring the use of deoxyribonucleic acid, DNA, as an information storage medium. DNA is an appealing candidate storage medium due to its small size, high information storage capacity, and decreasing cost in nucleic acid synthesis and sequencing. However, DNA by itself is sensitive to degradation by agents such as nucleases ubiquitous in the environment. As such, there is a need to develop materials for use as protective carriers of DNA barcodes.

### SUMMARY OF THE INVENTION

The present disclosure provides data storage systems and methods of making thereof. In some embodiments, the present disclosure provides engineered porous protein crystals that bind and/or adsorb guest information storage mediums systems and methods of making thereof. In some embodiments, the present disclosure provides tracking systems for organisms and methods of making thereof.

In certain embodiments, a data storage system herein may comprise an engineered host porous protein crystal and at least one guest molecule. In some embodiments, a data storage system herein may comprise an engineered host porous protein crystal and at least one guest molecule, wherein the engineered host porous protein crystal may comprise at least one pore having a diameter equal to or greater than 3 nm. In some embodiments, a data storage system herein may comprise an engineered host porous protein crystal and at least one guest molecule, wherein the engineered host porous protein crystal may comprise at least one pore having a diameter equal to or greater than 3 nm wherein the at least one pore's diameter may be large enough to permit entry of the entirety of at least one guest molecule into the pore, and wherein the at least one guest molecule may comprise at least one guest information storage medium and may be adsorbed within the engineered host porous protein crystal. In some embodiments, a data storage system herein may further comprise at least one binding site for the at least one guest molecule. In some embodiments, a data storage system herein may further comprise at least one binding site for the at least one guest molecule within the interior of the at least one pore.

In some embodiments, a data storage system herein may comprise at least one guest information storage medium comprised of guest deoxyribonucleic acid (DNA). In some embodiments, a data storage system herein may comprise guest DNA wherein the guest DNA may comprise at least one abiotic DNA sequence. In some embodiments, a data storage system herein may comprise a guest DNA that may be comprised of at least one engineered DNA sequence. In some embodiments, a data storage system herein may comprise a guest DNA that may be comprised of at least one engineered DNA sequence, wherein the at least one engineered DNA sequence may comprise a synthetic barcode sequence.

In some embodiments, a data storage system herein may comprise at least one guest information storage medium that may comprise at least one modular barcode library. In some embodiments, a data storage system herein may comprise at least one modular barcode library wherein the at least one modular barcode library may comprise oligonucleotide blocks. In some embodiments, a data storage system herein may comprise at least one modular barcode library wherein the at least one modular barcode library may comprise at least four oligonucleotide blocks. In some embodiments, a data storage system herein may comprise at least one modular barcode library wherein the at least one modular barcode library may comprise at least four oligonucleotide blocks, wherein an oligonucleotide block may comprise a single-stranded DNA overhang complementary to an adjacent oligonucleotide block.

In some embodiments, a data storage system herein may comprise oligonucleotide blocks that may be assembled into modular barcode libraries. In some embodiments, a data storage system herein may comprise oligonucleotide blocks that may be assembled into modular barcode libraries in equimolar amounts. In some embodiments, a data storage system herein may comprise at least four oligonucleotide blocks that may be assembled into modular barcode libraries. In some embodiments, a data storage system herein may comprise at least four oligonucleotide blocks that may be assembled into modular barcode libraries in equimolar amounts.

In some embodiments, a data storage system herein may comprise at least one modular barcode library comprising at least about 5 base pairs (bp). In some embodiments, a data storage system herein may comprise at least one modular barcode library comprising about 5 bp to about 300 bp.

In some embodiments, a data storage system herein may comprise at least one modular barcode library comprising at least about 50 unique barcode sequences. In some embodiments, a data storage system herein may comprise at least one modular barcode library comprising about 50 to about 500 unique barcode sequences.

In some embodiments, a data storage system herein may comprise at least one guest information storage medium that may be recovered from the engineered host porous protein crystal. In some embodiments, a data storage system herein may comprise guest DNA that may be recovered from the engineered host porous protein crystal. In some embodiments, a data storage system herein may comprise guest DNA that may be released from the engineered host porous protein crystal after incubating the crystal in a mixture comprising dNTPs, ATP, or any combination thereof. In some embodiments, a data storage system herein may comprise at least one guest information storage medium that may comprise at least one engineered DNA sequence comprising a synthetic barcode sequence, wherein information encoded in the synthetic barcode sequence may be detected using PCR, qPCR, ddPCR, rtPCR, next-generation sequencing, or any combination thereof.

In certain embodiments, methods of generating a data storage system herein may comprise obtaining at least one engineered host porous protein crystal and incubating the engineered host porous protein crystal with at least one guest molecule to produce a porous protein crystal guest molecule conjugate. In some embodiments, methods of generating a data storage system herein may comprise obtaining an engineered host porous protein crystal, wherein the engineered host porous protein crystal may have been reacted with a crosslinking agent to produce a crosslinked porous protein crystal; and incubating the crosslinked porous protein crystal with at least one guest molecule to produce a porous protein crystal guest molecule conjugate, wherein the least one guest molecule may comprise at least one guest information storage medium.

In some embodiments, methods of generating a data storage system herein may comprise incubation with the least one guest information storage medium, wherein the at least one guest information storage medium may comprise at least one modular barcode library. In some embodiments, methods of generating a modular barcode library herein may comprise constructing oligonucleotide blocks from a pool of oligonucleotides; mixing the oligonucleotide blocks together; and, subjecting the mixture to heating, followed by annealing, and then ligation. In some embodiments, methods of generating a modular barcode library herein may comprise constructing at least four oligonucleotide blocks from a pool of oligonucleotides, wherein an oligonucleotide block may comprise a single-stranded DNA overhang complementary to an adjacent oligonucleotide block; mixing the at least four oligonucleotide blocks together in equimolar amounts; and, subjecting the mixture to heating, followed by annealing, and then ligation.

In some embodiments, methods of generating a data storage system herein may optionally further comprise methods for releasing least one guest molecule from the porous protein crystal guest molecule conjugate. In some embodiments, methods of releasing least one guest molecule from the porous protein crystal guest molecule conjugate herein may comprise incubating the porous protein crystal guest molecule conjugate in a mixture of dNTPs, ATP, or any combination thereof. In some embodiments, methods of generating a data storage system herein may optionally further comprise methods for recovery of a guest molecule released from a porous protein crystal guest molecule conjugate. In some embodiments, methods of recovering at least one guest molecule released from a porous protein crystal guest molecule conjugate may comprise use of PCR, qPCR, next-generation sequencing, or any combination thereof.

In certain embodiments, tracking systems for organisms herein may comprise at least one synthetic library and a porous protein crystal. In some embodiments, tracking systems herein may comprise a synthetic library encoded with unique barcode DNA sequences, and a crosslinked porous protein crystal, wherein the synthetic library may be stored in the crosslinked porous protein crystal. In some embodiments, tracking systems herein may comprise a synthetic next-generation sequencing (NGS) library. In some embodiments, tracking systems herein may comprise a synthetic library comprising at least one modular barcode library.

In some embodiments, tracking systems herein may comprise at least one modular barcode library comprised of oligonucleotide blocks. In some embodiments, tracking systems herein may comprise at least one modular barcode library comprised of at least four oligonucleotide blocks. In some embodiments, tracking systems herein may comprise at least one modular barcode library comprised of at least four oligonucleotide blocks, wherein an oligonucleotide block may comprise a DNA overhang complementary to an adjacent oligonucleotide block.

In some embodiments, tracking systems herein may comprise at least one modular DNA barcode that may comprise at least about 5 bp. In some embodiments, tracking systems herein may comprise at least one modular DNA barcode that may comprise about 5 bp to about 300 bp.

In some embodiments, tracking systems herein may comprise at least one modular DNA barcode that may comprise at least about 50 unique barcode sequences. In some embodiments, tracking systems herein may comprise at least one modular DNA barcode that may comprise about 50 to about 500 unique barcode sequences.

In some embodiments, tracking systems herein may be used in an organism wherein the organism may be algae, bacteria, plants, insects, fish, amphibians, reptiles, birds, and/or mammals. In some embodiments, tracking systems herein may be used in an organism wherein the organism may be an insect.

In some embodiments, tracking systems herein may mark at least one organism with at least one unique barcode DNA within a crosslinked porous protein crystal comprising the synthetic library as disclosed herein. In some embodiments, tracking systems herein may mark at least one insect with at least one unique barcode DNA within a crosslinked porous protein crystal comprising the synthetic library as disclosed herein. In some embodiments, tracking systems herein may mark at least one insect with at least one unique barcode DNA after ingestion of the crosslinked porous protein crystal comprising the synthetic library as disclosed herein. In some embodiments, tracking systems herein may mark at least one insect with at least one unique barcode DNA, wherein the crosslinked porous protein crystal comprising the synthetic library may be ingested by the at least one insect when the insect is a larva, pupa, adult, or any combination thereof.

In some embodiments, a synthetic library of a tracking system herein may be recovered from the crosslinked porous protein crystal with less than about 10% degradation. In some embodiments, a synthetic library of a tracking system herein may be recovered from the crosslinked porous protein crystal can may be subjected to PCR, qPCR, ddPCR, rtPCR, next-generation sequencing, or any combination thereof to determine the unique barcode DNA for the organism.

In some embodiments, tracking systems herein may comprise at least one synthetic library that may comprise at least about 10 million reads of the unique barcode DNA. In some embodiments, tracking systems herein may comprise at least one synthetic library that may comprise about 10 million to about 200 million reads of the unique barcode DNA.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure, which can be better understood by reference to the drawing in combination with the detailed description of specific embodiments presented herein.

FIGS. 1A-1D depict representative images of porous materials. FIG. 1A shows Campylobacter jejuni (CJ) crystals in glass dish well. FIG. 1B shows a schematic depicting the P622 space group, demonstrating the large axial pores present throughout the crystals. FIG. 1C shows guest DNA comprising 8, 15 or 125 basepairs (bp).

FIG. 1D shows a schematic of an experimental overview of recording and measuring the loading of guest DNA into host crystals.

FIGS. 2A-2C depict representative images of DNA loading into CJ crystals. FIG. 2A shows crystal orientation relative to objective lens during imaging. FIG. 2B shows confocal loading datasets with FAM-labeled 8mer guest for two separate crystals. FIG. 2C shows confocal loading datasets with FAM-labeled 15mer guest for two separate crystals.

FIGS. 3A-3C depict representative images of 125mer DNA loading into CJ crystals. Confocal images (DIC (FIG. 3A) and 488 nm (FIG. 3B)) of crystal following incubation in FAM-labeled 125mer are shown. FIG. 3C shows an agarose gel image of 125mer recovered from crystals following dNTP triggered release.

FIG. 4 depicts a representative graph of qPCR Recovery where plotted is the standard curve obtained from serial dilutions of previously amplified and purified 125mer. The red triangle represents the solution 125mer amount before ATP incubation. The black outlined triangle represents the solution 125mer amount following ATP incubation showing the ATP triggered release of guest DNA over a 12-hour period.

FIG. 5 depicts a representative image of modular barcode design and assembly.

FIGS. 6A-6B depict representative images of library design cost efficiency. FIG. 6A shows a table comparing estimated synthesis cost for 1 and 256 barcodes, separately, between the conventional approach and this work. FIG. 6B shows a comparative bar chart showing the increasing estimated oligo synthesis cost per the conventional approach of purchasing pre-made oligos of the desired length.

FIG. 7 depicts a representative image of powercode design. Top: The number of barcodes combined, k, determines the number of possible powercodes that can be created from the 256 modular barcodes. Middle: each block represents a unique barcode strand that is designated with an index value. Shaded blocks represent the specific dsDNA barcodes included within a particular powercode subset. Bottom: As the number of barcodes per powercode increases, the available pool of samples containing a unique.

FIG. 8 depicts a representative image of a schematic for the experimental design for Culex tarsalis microcrystal exposure.

FIG. 9 depicts a representative image of a Kaplan-Meier curve of Cx. tarsalis larvae's successful pupation probability after exposure to microcrystal-laden or control Feeds during development.

FIGS. 10A-10E depict representative images of microcrystal detection in the digestive tract of fourth instar larvae. FIG. 10A is an image from control larvae, which received no microcrystals and shows no red fluorescence. FIGS. 10B-10D were taken from treatment larvae, which indicated markedly brighter fluorescence and the presence of crystals at different portions of the gastrointestinal tract (FIG. 10B=foregut, FIG. 10C=midgut, and FIG. 10D=hindgut). FIG. 10E is a schematic of a larvae where encircled areas correspond to the area of indicated images—FIGS. 10B-10D.

FIG. 11 depicts a representative image of a Kaplan-Meier Survival curve of Cx. tarsalis adults that were reared from microcrystal-laden or control larval development conditions.

FIGS. 12A-12B depict representative images of confocal imaging indicative of microcrystal transstadial transmission. FIG. 12A depicts images from dissected larvae gut. FIG. 12B depicts images from dissected gut of adult Cx. tarsalis where encircled is a Texas-red conjugated microcrystal found in the adult gut.

FIG. 13 depicts a representative image of an agarose gel of DNA-loaded crystals after incubation in the presence or absence of mosquito homogenate.

FIG. 14 depicts a representative image of DNA barcodes detected using qPCR and next generation sequencing (NGS). Analysis of the corresponding melt curves displays peaks overlapping with the estimated barcode Tm (˜80° C.) suggesting successful DNA barcode detection using qPCR.

FIG. 15 depicts a representative image of NGS coverage results for a single modular barcode, as determined using the software Geneious Prime for the top 100 reads, which displayed markedly high coverage across the entire barcode sequence, including the variable 6nt regions shaded in gray.

FIG. 16 depicts a representative image of a histogram of NUPACK scoring results for each of the ˜3,600 candidates. The image depicts a bell-shaped distribution with an average of approx. −181 kcal/mol describing the propensity of each candidate, comprised of 8 single-stranded oligonucleotides, to form the target secondary structure. The top 4 scoring candidates were chosen for experimental validation.

FIGS. 17A-17B depict representative images of crystal prevalence in emerged male mosquitoes. Barcode was amplified from adult male mosquitoes reared on (fed) barcode-laden microcrystals as larvae. qPCR amplification plots show barcode amplification from mosquito samples between 24-32 cycles (FIG. 17A). Positive controls represent serial dilutions of naked barcode. Negative controls represent PCR master mix with no template added. Each well contained one homogenized male mosquito (FIG. 17B).

FIG. 18 depicts a representative image of melt curves for qPCR amplification of samples recovered from laboratory and field mosquitoes. Groups of emerged adult mosquitoes=SR1-SR3; groups of wild-caught adults=FR1-FR3; groups of adults reared in the laboratory=LR1-LR3.

FIG. 19 depicts a representative image of standard gel electrophoresis of the synthetic barcode amplicons, revealing a major peak at the 84-bp position, and a variable minor population of greater size.

FIG. 20 depicts a representative graph of the size distribution of 1 million aligned NGS reads for samples extracted from the larger electrophoresis bands in FIG. 19.

FIG. 21 depicts a representative image of the sequence of intended barcode amplicon, and the sequence and schematic view of the longer off-target amplicons that are also derived from synthetic barcode DNA sequences.

FIGS. 22A-22C depicts representative images of modular barcodes synthesized by mixing, annealing and ligating 8 single-stranded oligonucleotides to form the core barcode (FIG. 22A) comprised of the Source Tag region flanked by two regions of constant sequence shared among all modular barcode variants. Following Source Tag construction, several rounds of overhang PCR are performed to append the unique Trap Tag sequence (FIG. 22B), and the illumine adapter sequences necessary for Input to Next-Generation Sequencing (NGS) platforms (FIG. 22C).

FIG. 23 depicts a representative image of a modular barcode index resulting from modular barcode experimental validation.

### DETAILED DESCRIPTION OF THE INVENTION

The present disclosure may be understood by reference to the following detailed description, taken in conjunction with the drawings as described above.

The growing demand for data storage has driven the need for research into alternative materials for information storage mediums. Several inherent properties of DNA contribute to it serving as an information storage medium including, but not limited to, high encoded information density, stability and virtually guaranteed access to the requisite machinery for writing and reading DNA. However, DNA by itself is sensitive to degradation by agents such as nucleases ubiquitous in the environment. If protected within tough protected carrier particles that were ultimately biodegradable, DNA could become the barcode material of choice.

The present disclosure is based in part on the discovery that highly porous, cross-linked protein crystals can be used for storing and protecting barcode DNA. Disclosed herein are data showing that using protein crystals as part of a data storage system protected barcode DNA from degradation. Data storage systems of the present disclosure comprise a modular DNA library encompassing interchangeable ‘blocks’ with multiple variants for increasing the number of barcode sequences possible from a handful (e.g., at least about 4 to at least about 30) of oligonucleotides. DNA barcode-loaded protein crystals disclosed herein may possess an elevated resistance against degradation, allowing for use in marking organisms (e.g., insects). Accordingly, the present disclosure provides data storage systems and methods of making thereof, as well as DNA barcode-loaded protein crystals and methods of making thereof.

### I. Definitions

As used herein, the terms “about” and “approximately” designate that a value is within a statistically meaningful range. Such a range can be typically within 20%, more typically still within 10%, and even more typically within 5% of a given value or range. The allowable variation encompassed by the terms “about” and “approximately” depends on the particular system under study and can be readily appreciated by one of ordinary skill in the art.

When introducing elements of the present disclosure or the preferred embodiments(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The term “conjugate,” as used herein refers to guest molecules that are entrapped, non-covalently bound, or covalently bound to a porous protein crystal.

“Nucleic acid sequence”, as used herein, refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group. Nucleic acid sequences may be used herein to refer to oligonucleotides, or polynucleotides, and fragments or portions thereof, and to DNA or RNA of genomic or synthetic origin that may be single- or double-stranded and represent the sense or antisense strand. A nucleic acid sequence can refer to a succession of bases signified by a series of a set of five different letters corresponding to a DNA (using GACT) or an RNA (GACU) molecule.

### II. Data Storage Systems

A data storage system refers to any medium capable of recording and/or preserving digital information for ongoing or future operations. For example, a digital data storage system can encode text, photos, or any other kind of information as a series of 0s and 1s. In some embodiments, a data storage system herein may be a biological data storage system. In some embodiments, a data storage system herein may be a biological data storage system wherein the medium may be a synthetic nucleotide sequence. In some embodiments, a data storage system herein may be a biological data storage system wherein the medium may be DNA. Without being bound by theory, a biological data storage system of the present disclosure may record text, photos, or any other kind of information in a DNA medium wherein the information encoded in the DNA uses the four nucleotides that make up the genetic code: A, T, G, and C. For example, G and C could be used to represent 0 while A and T represent 1.

In some embodiments, a data storage system herein may comprise an information storage medium in a protective material. In some embodiments, a data storage system herein may comprise an information storage medium in an engineered host porous protein crystal. In some embodiments, a data storage system herein may comprise a biological medium in an engineered host porous protein crystal. In some embodiments, a data storage system herein may comprise a synthetic nucleotide sequence as a medium in an engineered host porous protein crystal. In some embodiments, a data storage system herein may comprise DNA as a medium in an engineered host porous protein crystal.

In some embodiments, a data storage system herein may comprise a synthetic nucleotide sequence as a medium in an engineered host porous protein crystal wherein the synthetic nucleotide sequence can be extracted from the engineered host porous protein crystal. In some embodiments, a data storage system herein may comprise DNA as a medium in an engineered host porous protein crystal wherein the DNA can be extracted from the engineered host porous protein crystal.

In some embodiments, a data storage system herein may comprise an engineered host porous protein crystal and at least one guest molecule, wherein the at least one guest molecule comprises at least one guest information storage medium and is adsorbed within the engineered host porous protein crystal. In some embodiments, a data storage system herein may comprise an engineered host porous protein crystal and at least one guest synthetic nucleotide sequence, wherein the at least one guest synthetic nucleotide sequence can serve as an information storage medium and can be adsorbed within the engineered host porous protein crystal. In some embodiments, a data storage system herein may comprise an engineered host porous protein crystal and at least one guest DNA, wherein the at least one guest DNA can serve as an information storage medium and can be adsorbed within the engineered host porous protein crystal.

### III. Engineered Host Porous Protein Crystal

In some embodiments, the present disclosure provides data storage system comprising at least an engineered host porous protein crystal. The terms “engineered host porous protein crystal” and “porous protein crystals” are used interchangeably throughout the present disclosure. In accordance with some embodiments herein, the present disclosure provides porous protein crystals. Some embodiments of the present disclosure provide a 3-dimensional porous protein crystal. In accordance with these embodiments, a porous protein crystal herein may comprise at least one protein monomer that assembles to form multiple unit cells, with each unit cell capable of hosting at least one guest molecule.

### (a) Protein Identity

In certain embodiments, a porous protein crystal comprises a protein. Proteins that are able to crystalize into a protein scaffold with an appropriate pore size are known by those of skill in the art. A person skilled in the art would be able to inspect the known crystal packing arrangement for proteins deposited in the Research Collaboratory for Structural Bioinformatics Protein Data Bank (RCSB PDB) (See, e.g., Berman et al. Nucleic Acids Research, 2000; 28(1):235-242, herein incorporated by reference in its entirety). A person skilled in the art could then select a protein crystal known to crystallize into a protein scaffold with an appropriate pore size.

In some embodiments, the protein may be the NHR2 domain of the fusion protein AML1-ETO from Homo sapiens, chloramphenicol phosphotransferase from Streptomyces venezuelae, gastric lipase from Homo sapiens, a Bro1 domain containing protein Brox from Homo sapiens, a putative cell adhesion protein (BACOVA_04980) from Bacteroides ovatus, glycoprotein 1 b from Homo sapiens, an arginine decarboxylase SpeA from Campylobacter jejuni, a cystathionine beta-synthase from Homo sapiens, a (+)-bornyl diphosphate synthase from Salvia officinalis, a measles virus hemagglutinin bound to its cellular receptor SLAM (form 1) from Saguinus oedipus, an invertase 2 from Saccharomyces cerevisiae, a putative periplasmic YCEI-like protein from Campylobacter jejuni, an atrial natriuretic peptide clearance receptor from Homo sapiens, a catalytic domain of transaminase PigE from Serratia sp. fs14, a putative glycosidase from Thermotoga maritima, a sorting nexin 10 from Homo sapiens, photosystem I from Synechococcus elongatus, lysostaphin from Staphylococcus simulans, Pyk2 (proline-rich tyrosine kinase 2) in complex with paxillin from Gallus gallus, an Insulin degrading enzyme from Homo sapiens, an artocarpin from Artocarpus integer, a neuropilin-1 extracellular domains from Mus musculus, a tryptophanyl-tRNA synthetase from Saccharomyces cerevisiae, DNA topoisomerase II from Escherichia coli, a V delta 1 T Cell Receptor in complex with antigen-presenting glycoprotein CD1d from Homo sapiens, a Mus musculus antibody-bound Homo sapiens Prolactin receptor, a fructose 1-6-bisphosphate aldolase from Homo sapiens, a core fragment from unphosphorylated STAT3 (signal transducer and activator of transcription 3) from Mus musculus, a fusion glycoprotein FO from Human metapneumovirus and neutralizing antibody DS7 from Homo sapiens, a growth-arrest-specific protein 6 precursor and tyrosine-protein kinase receptor UFO from Homo sapiens, a Sas-6 cartwheel hub from Leishmania major, a neuraminidase from Influenza a virus, a molybdopterin-guanine dinucleotide biosynthesis protein B from Escherichia coli, an apical membrane antigen AMA1 and putative Rhoptry neck protein 2 from Eimeria tenella, a complex between NADPH-cytochrome P450 reductase and heme oxygenase 1 from Rattus norvegicus, a proprotein convertase subtilisin/kexin type 9 in complex with low-density lipoprotein receptor from Homo sapiens, a major tropism determinant P1 in complex with pertactin extracellular domain from Bordetella bronchiseptica and Bordetella virus bpp1.

In some embodiments, the protein may be a constituent of the following Protein Data Bank entries: 3FOQ, 4JOL, 409X, 1QHN, 1R5U, 1S49, 3S4Z, 3AL8, 2BDM, 3C3E, 3EN1, 1 IVI, 1 MHP, 3RIP, 1 EA0, 4FHM, 3GB8, 1HLG, 4O5I, 3R9M, 3ZXU, 3ABS, 1S4F, 3UF1, 1V3D, 1WCM, 4CNI, 3Q17, 3RZI, 2BE5, 1GWB, 4MNA, 3NZP, 1OGP, 3FCU, 3K7A, 4L3V, 1N21, 4U7P, 3ALZ, 1RLR, 4EQV, 2FGS, 1JDN, 4MQ9, 4 PPM, 3QZ2, 3WOD, 2AAM, 4AY5, 4IWO, 3K1F, 4PZG, 3PCQ, 2QUK, 3RJ1, 3W3A, 3ALW, 4AY6, 4LXC, 405J, 4R32, 2WBY, 1ZBU, 3A5C, 4J23, 4AVT, 1TYE, 1VBP, 4GZ9, 4WJW, 4C8Q, 2YHB, 3DQQ, 3KT8, 1 D6M, 4MNG, 2TMA, 4I18, 1QO5, 3CWG, 4DAG, 3D38, 2C5D, 4CKP, 3CL2, 1P9N, 4YIZ, 3WKT, 3P5C, and 2IOU.

In some embodiments, the protein may be a YCEI protein from Campylobacter jejuni, a pyridine nucleotide-disulfide family oxidoreductase from Enterococcus faecalis, a major tropism determinant P1 in complex with pertactin extracellular domain from Bordetella bronchiseptica and Bordetella virus bpp1, a putative cell adhesion protein (BACOVA_04980) from Bacteroides ovatus, Pyk2 (proline-rich tyrosine kinase 2) in complex with paxillin from Gallus gallus, and the NHR2 domain of the fusion protein AML1-ETO from Homo sapiens.

### (b) Protein Crystallization

In certain embodiments, the protein is crystallized to form a porous protein crystal. The porous protein crystal comprises multiple unit cells.

In general, the protein may be crystallized using standard techniques in the field. Further, the method can and will vary depending on the identity of the protein. Suitable methods include, without limit, vapor diffusion, sitting drop, hanging drop, counter-diffusion, batch, microbatch, microdialysis, free-interface diffusion, and seeding (See, e.g., Weber, Methods Enzymology, 1997; 276:13-22, herein incorporated by reference in its entirety).

Briefly, protein crystallization is influenced by purities and concentrations of the protein, the types and concentrations of protein crystallization agents, pH conditions, temperature conditions, etc. Therefore, protein crystallization conditions are determined according to a combination of these parameters. Specifically, screening of protein crystallization conditions refers to selecting, from the multiple combinations of the parameters above, the combination of parameters suitable for crystallization of a target protein. Protein crystallization conditions are reported for structures present in the PDB. Thus, a person skilled in the art would be able to recapitulate known protein crystal forms by conducting crystallization experiments that emulate the published conditions.

### (c) Protein Crystal Pore Diameter

In certain embodiments, the porous protein crystal comprises a plurality of pores or solvent channels. These pores or solvent channels allow for entry of the guest molecule into the porous protein crystal. Once the guest molecule has entered the porous protein crystal it may then bind to at least one binding site within the pore of the porous protein crystal. The pores should be an appropriate size to allow entry of the guest molecule.

In some embodiments, the porous protein crystal may have a pore diameter of from about 3 nm to about 50 nm. In some embodiments, the pore diameter may be about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, or about 50 nm. In additional embodiments, the pore diameter may be equal to or greater than about 4 nm, equal to or greater than about 5 nm, equal to or greater than about 6 nm, equal to or greater than about 7 nm, equal to or greater than about 8 nm, equal to or greater than about 9 nm, equal to or greater than about 10 nm, equal to or greater than about 11 nm, equal to or greater than about 12 nm, equal to or greater than 13 nm, equal to or greater than about 14 nm, equal to or greater than about 15 nm, equal to or greater than about 16 nm, equal to or greater than about 17 nm, equal to or greater than about 18 nm, equal to or greater than about 19 nm, equal to or greater than about 20 nm, equal to or greater than about 21 nm, equal to or greater than about 22 nm, equal to or greater than about 23 nm, equal to or greater than about 24 nm, equal to or greater than about 25 nm, equal to or greater than about 26 nm, equal to or greater than about 27 nm, equal to or greater than about 28 nm, equal to or greater than about 29 nm, or equal to or greater than about 30 nm.

In some embodiments, the plurality of pores may have an average diameter of from about 3 nm to about 50 nm. In some embodiments, the plurality of pores may have an average diameter of about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, or about 50 nm. In additional embodiments, the plurality of pores may have an average diameter equal to or greater than about 4 nm, equal to or greater than about 5 nm, equal to or greater than about 6 nm, equal to or greater than about 7 nm, equal to or greater than about 8 nm, equal to or greater than about 9 nm, equal to or greater than about 10 nm, equal to or greater than about 11 nm, equal to or greater than about 12 nm, equal to or greater than 13 nm, equal to or greater than about 14 nm, equal to or greater than about 15 nm, equal to or greater than about 16 nm, equal to or greater than about 17 nm, equal to or greater than about 18 nm, equal to or greater than about 19 nm, equal to or greater than about 20 nm, equal to or greater than about 21 nm, equal to or greater than about 22 nm, equal to or greater than about 23 nm, equal to or greater than about 24 nm, equal to or greater than about 25 nm, equal to or greater than about 26 nm, equal to or greater than about 27 nm, equal to or greater than about 28 nm, equal to or greater than about 29 nm, or equal to or greater than about 30 nm.

### (d) Protein Binding Site

In certain embodiments, the porous protein crystal comprises at least one binding site within a pore to allow at least one guest molecule to bind. In certain embodiments, the porous protein crystal comprises at least one binding site within a pore to allow at least one guest molecule to bind and be adsorbed within an engineered host porous protein crystal disclosed herein. In an embodiment, the at least one binding site may be an amino acid, a chemically modified amino acid, a proximal collection of amino acids, a peptide sequence, or combinations thereof.

In some embodiments, the protein binding site may be designed so the binding between it and the guest molecule is reversible. In other words, the guest molecule may be released from the binding site. Release from the binding site may result when the porous protein crystal guest molecule conjugate is exposed to a specific condition (e.g., solvent, temperature, light, electric field, magnetic field, etc.). By way of a non-limiting example, the guest molecule may be a nanoparticle that may be released from the porous protein crystal by exposure to a solvent, which breaks the specific porous protein/nanoparticle interaction.

(i) Naturally Occurring Amino Acids

In some embodiments, the at least one binding site may be an amino acid. In some embodiments, the amino acid may be histidine and cysteine. Other canonical amino acids may be selectively modified by a variety of reagents. Examples of modifying agents are provided in Hermanson, G. T. Bioconjugate Techniques. (Academic Press, 2013), herein incorporated by reference in its entirety. The at least one binding site may be engineered or modified (e.g., substitution mutation) to be at a specific location within the pore to direct the guest molecule to occupy a specific location with the pore.

(ii) Non-Canonical Amino Acids

In some embodiments, the at least one binding site may be a non-canonical amino acid. In some embodiments, the non-canonical amino acids would be capable of “click chemistry.” Suitable non-canonical amino acids may comprise, but are not limited to, akynes, azides, or tetrazines.

(iii) Chemically Modified Amino Acids

In some embodiments, the at least one binding site may be a chemically modified amino acid. Suitable amino acids for chemical modification may include cysteine, lysine, histidine, tyrosine, serine, arginine, aspartic acid, glutamic acid, and tryptophan. In some embodiments, the amino acid may be modified by a modifying agent.

Suitable modifying agents may include, without limit, Ellman's reagent (i.e., 5,5′-Disulfanediylbis(2-nitrobenzoic acid)), tetrathionate, selenocystine, hydroxymercuribenzoate (MBO), monobromobimane (mBBr), dibromobimane (dBBr), dibromomaleimide (dBM), N-substituted dibromomaleimides (R-dBM, wherein R may be any functionalization of the dibromomaleimide), p-toluenesulfonyl chloride (TosCl), succinimidyl iodoacetate (SIA), N-succinimidyl S-acetylthioacetate (SATA), (succinimidyl 3-(2-pyridyldithio)propionate (SPDP), N-α-maleimidoacet-oxysuccinimide ester (AMAS), or 1-Ethyl-3-[3-dimethylaminopropyl] carbodiimide hydrochloride (EDC). Additional modifying agents are provided in Hermanson, G. T. Bioconjugate Techniques. (Academic Press, 2013), herein incorporated by reference in its entirety.

(iv) Peptide Sequence

In some embodiments, the at least one binding site may be a peptide sequence with known affinity for another biological polymer. In some embodiments, the peptide sequence may comprise one portion of a split protein, one member of an oligomeric complex, a sequence with binding affinity for DNA, or a sequence with a binding affinity for a nanoparticle.

In some embodiments, the at least one binding site may be a metal-affinity peptide sequence. In an exemplary embodiment, the metal-affinity peptide sequence may be a histidine tag. In an additional exemplary embodiment, the histidine tag may be a C-terminal histidine tag or an N-terminal histidine tag. In some embodiments, the histidine tag may comprise from 2 histidine residues to about 10 histidine residues. In an exemplary embodiment, the histidine tag may comprise 6 histidine residues.

In some embodiments, the metal-affinity peptide sequence may bind a metal ion. Suitable metal ions include, without limit, Ni, Cu, Zu, Fe, and Co. In an exemplary embodiment, the metal ion may be Ni. In another exemplary embodiment, the metal ion may be Zn.

(v) Location of the Binding Site

In some embodiments, the position of the at least one binding site within the porous protein crystal pore can and will vary depending on the desired location of the at least one guest molecule within the porous protein crystal pore. A person skilled in the art would be able to select the appropriate location of the at least one binding site within the porous protein crystal pore to direct the at least one guest molecule to be at a specific location within the porous protein crystal pore.

### (e) Protein Stability

In some embodiments, the porous protein crystal may be stabilized by forming covalent bonds, non-covalent bonds, or combinations thereof between amino acids present in adjacent monomers. A stabilized porous protein crystal will be more stable than an un-stabilized porous protein crystal if transferred to solution conditions that differ from the crystal growth mother liquor. In some embodiments, a stabilized protein crystal grown in high salt conditions, may persist when transferred to low salt conditions. Some benefits associated with increased stability include, but are not limited to, allowing for a high quality of diffraction, providing macroscopic crystal stability, and rendering the porous protein crystal competent for guest loading and release.

(i) Covalent Bonds

In some embodiments, covalent bonds may be formed by reacting amino acids present in adjacent monomers with a crosslinking agent. In some embodiments, covalent bonds may be formed by reacting homogenous or heterogeneous amino acids present in adjacent monomers with a crosslinking agent. In some embodiments, covalent bonds may be formed between two sulfhydryl containing amino acids. In some embodiments, covalent bonds may be formed between two amine containing amino acids. In some embodiments, covalent bonds may be formed between an amine containing amino acid and a sulfhydryl containing amino acid. In some embodiments, covalent bonds may be formed between an amine containing amino acid and a carboxylate containing amino acid.

Suitable crosslinking agents may include, without limit, aldehydes, bis-NHS esters, bis-imidoesters, bis-maleimides, bis-haloalkyls, or carbodiimide reactive compounds; and combinations thereof.

Suitable aldehyde crosslinking agents may include, without limit, glutaraldehyde, formaldehyde, glyoxal, and combinations thereof.

Suitable NHS ester crosslinking agents will include 2 or more NHS ester groups, separated by linkers that may include 1-13 atoms, which may include, without limit, N,N′-Disuccinimidyl carbonate; N,N′-Disuccinimidyl oxalate; sulfodisuccinimidyl tartrate (Sulfo-DST); 3,3′-dithiobis[sulfosuccinimidylpropionate](DTSSP); bis(sulfosuccinimidyl)suberate (BS3); ethylene glycol bis[sulfosuccinimidylsuccinate] (Sulfo-EGS); and combinations thereof.

Suitable bis-imidoesters crosslinking agents may include, without limit, dithiobispropionimidate (DTBP), dimethyl adipimidate (DMA), and combinations thereof.

Suitable bis-maleimide crosslinking agents may include, without limit, 1,4-bismaleimidobutane; 1,8-bismaleimido-diethyleneglycol; 1,11-bismaleimido-triethyleneglycol; bismaleimidohexane; bismaleimidoethane; dithiobismaleimidoethane; and combinations thereof.

Suitable bis-haloalkyl crosslinking agents may include, without limit, dibromobimane; dibromomaleimide; N-substituted dibromomaleimides; dibromoxylene; phosgene; dichloroethane; and combinations thereof.

Suitable carbodiimide crosslinking agents may include, without limit, 1-Ethyl-3-[3-dimethylaminopropyl] carbodiimide hydrochloride (EDC); N′,N′-dicyclohexyl carbodiimide (DCC); N,N′-diisopropylcarbodiimide (DIC); and combinations thereof.

In some embodiments, the crosslinking agent may be 1-Ethyl-3-[3-dimethylaminopropyl] carbodiimide hydrochloride (EDC); formaldehyde; formaldehyde and urea; formaldehyde and guanidinium hydrochloride; glyoxal; glyoxal and dimethylamine borane (DMAB); glutaraldehyde; glutaraldehyde and dimethylamine borane complex; 1-Ethyl-3-[3-dimethylaminopropyl] carbodiimide hydrochloride (EDC); 1-Ethyl-3-[3-dimethylaminopropyl] carbodiimide hydrochloride (EDC) and imidazole; 1-Ethyl-3-[3-dimethylaminopropyl] carbodiimide hydrochloride (EDC) and sulfo N-hydroxysulfosuccinimide (sulfo-NHS); 1-Ethyl-3-[3-dimethylaminopropyl] carbodiimide hydrochloride (EDC), sodium malonate, and hydroxysulfosuccinimide (sulfo-NHS).

In some embodiments, the crosslinking agent may be contacted with the porous protein crystal from about 5 minutes to about 24 hours. In some embodiments, the crosslinking agents may be contacted with the porous protein crystal for about 5 minutes, about 10 minutes, about 20 minutes, about 30 minutes, about 40 minutes, about 50 minutes, about 60 minutes, 1.5 hours, about 2 hours, about 2.5 hours, about 3 hours, about 3.5 hours, about 4 hours, about 4.5 hours, or about 5 hours, about 5.5 hours, about 6 hours, about 6.5 hours, about 7 hours, about 7.5 hours, about 8.5 hours, about 9 hours, about 9.5 hours, about 10 hours, about 10.5 hours, about 11 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, or about 24 hours.

In some embodiments, the crosslinking agent may be contacted with the porous protein crystal from about 5 minutes to about 24 hours. In some embodiments, the crosslinking agents are contacted with the porous protein crystal about 5 minutes, about 10 minutes, about 20 minutes, about 30 minutes, about 40 minutes, about 50 minutes, about 60 minutes, 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours.

In some embodiments, the crosslinking may be reversible. In other embodiments the crosslinking may be irreversible.

The amount of crosslinking agent may and will depend upon the concentration of the porous protein crystal and the identity of the protein. A person of ordinary skill in the art would be able to select the appropriate amount and concentration of the crosslinking agent to produce a crosslinked porous protein crystal.

(ii) Non-Covalent Bonds

In some embodiments, non-covalent bonds may be formed between amino acids present in adjacent monomers. In some embodiments, the non-covalent bonds include electrostatic and hydrophobic interactions.

In some embodiments, electrostatic interactions may be between charged amino acids. In a further embodiment, electrostatic interactions may be between positively and negatively charged amino acids. Charged amino acids include aspartic acid, glutamic acid, lysine, arginine, and histidine. A person skilled in the art would be able to estimate the charge of the aforementioned amino acids based on the pH of the solvent or buffer.

In some embodiments, hydrophobic interactions may be between at least two hydrophobic amino acids. Hydrophobic amino acids include alanine, isoleucine, leucine, phenylalanine, valine, proline, and glycine.

### IV. Guest Molecule

In some embodiments, the present disclosure provides data storage system comprising at least one guest molecule. Some embodiments of the present disclosure provide at least one guest molecule that may bind to at least one binding side in the porous protein crystal pore. Some embodiments of the present disclosure provide at least one guest molecule that may be adsorbed within the engineered host porous protein crystal.

In some embodiments, a guest molecule herein may be a guest information storage medium. As used herein, a guest information storage medium may comprise a nanoparticle, a macromolecule, or a combination thereof suitable for the recording of information in the nanoparticle, the macromolecule, or the combination thereof.

In some embodiments, the at least one guest information storage medium may comprise a nanoparticle. Suitable nanoparticles may include transition metals, noble metals, or lanthanides. In some embodiments, the nanoparticle may have a diameter of about 3 nm to about 40 nm (e.g., about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm). In some embodiments, the nanoparticle may comprise more than about 25 metal atoms. In some embodiments, the nanoparticle may comprise about 25 metal atoms to about 400 metal atoms (e.g., about 25, about 30, about 35, about 40, about 45, about 50, about 55, about 60, about 65, about 70, about 75, about 80, about 85, about 90, about 95, about 100, about 125, about 150, about 175, about 200, about 225, about 250, about 275, about 300, about 325, about 350, about 375, about 400 metal atoms).

In some embodiments, the at least one guest information storage medium may comprise a macromolecule. In some embodiments, the at least one guest information storage medium may comprise synthetic and/or biological polymers. In some embodiments, the polymers may be ordered or disordered. In some embodiments, the polymers may be homogeneous or heterogeneous.

In some embodiments, the at least one guest information storage medium may comprise a biomacromolecule. Suitable biomacromolecules may include, without limit, an oligonucleotide (e.g., DNA or RNA) sequence, a polypeptide, a polysaccharide, or a polyphenol. In some embodiments, the at least one guest information storage medium may comprise at least one guest synthetic nucleic acid sequence. In some embodiments, the at least one guest information storage medium may comprise at least one guest DNA. In some embodiments, the guest DNA may comprise at least one abiotic DNA sequence. As used herein, abiotic DNA refers to synthesized DNA. It is understood herein that abiotic DNA does not include DNA extracted from non-engineered systems. In some embodiments, abiotic DNA herein is chemically synthesized DNA. In some embodiments, abiotic DNA herein is enzymatically synthesized DNA. In some embodiments, the guest DNA may comprise at least one engineered DNA sequence. In some embodiments, the guest DNA may be at least about 1 bp, at least about 4 bp, at least about 8 bp, or at least about 10 bp. In some embodiments, the guest DNA may be about 1 bp to about 300 bp. In some embodiments, the guest DNA may be about 1 bp to about 300 bps, about 2 bp to about 275 bps, about 3 bp to about 250 bps, about 4 bp to about 225 bps, about 5 bp to about 200 bps, about 6 bp to about 175 bps, about 7 bp to about 150 bps, or about 8 bp to about 125 bps.

In some embodiments, the guest molecule may comprise a synthetic barcode sequence. In some embodiments, the synthetic barcode sequence may be at least about 1 bp, at least about 4 bp, at least about 8 bp, or at least about 10 bp. In some embodiments, a synthetic barcode sequence may be about 1 bp to about 300 bp. In some embodiments, a synthetic barcode sequence may be about 1 bp to about 300 bps, about 2 bp to about 275 bps, about 3 bp to about 250 bps, about 4 bp to about 225 bps, about 5 bp to about 200 bps, about 6 bp to about 175 bps, about 7 bp to about 150 bps, or about 8 bp to about 125 bps.

In some embodiments, a synthetic barcode sequence comprises at least one oligonucleotide. In some embodiments, at least two oligonucleotides can be used to generate an oligonucleotide block. In accordance with some embodiments, an oligonucleotide block comprises a DNA overhang complementary to an adjacent oligonucleotide block. In some embodiments, at least about two oligonucleotides may be used to generate an oligonucleotide block. In some embodiments, about two oligonucleotides to about 12 oligonucleotides (e.g., about 2, 3, 4, 5, 6, 7, 8, 9, 10, 11, 12) can be used to generate an oligonucleotide block. In some embodiments, about four oligonucleotides can be used to generate an oligonucleotide block. In some embodiments, sequence specificity of each barcode may be achieved by the unique nucleotide sequence within each oligonucleotide block.

In some embodiments, a synthetic barcode sequence may further comprise at least one probe for detection. Non-limiting examples of detectable probes suitable for use herein can include a fluorophore (e.g., Texas red, Alexa 488), fluorescein, FAM, or any combination thereof.

In some embodiments, the guest molecule may comprise at least one modular barcode library. In accordance with these embodiments, a modular barcode library comprises at least one oligonucleotide block. In some embodiments, a modular barcode library may comprise at least about one, at least about two, or at least about four oligonucleotide blocks. In some embodiments, a modular barcode library may comprise about one oligonucleotide block to about 1000 oligonucleotide blocks, about one oligonucleotide block to about 500 oligonucleotide blocks, about one oligonucleotide block to about 100 oligonucleotide blocks, or about one oligonucleotide block to about 10 oligonucleotide blocks. In some embodiments, a modular barcode library may comprise about one oligonucleotide block, about two oligonucleotide blocks, about three oligonucleotide blocks, about four oligonucleotide blocks, about five oligonucleotide blocks, about six oligonucleotide blocks, about seven oligonucleotide blocks, about eight oligonucleotide blocks, about nine oligonucleotide blocks, or about ten oligonucleotide blocks.

In some embodiments, a modular barcode library may comprise an oligonucleotide block having a DNA overhang complementary to an adjacent oligonucleotide block. In some embodiments, oligonucleotide blocks comprising a modular barcode library herein may be arranged in tandem based on the presence of a DNA overhang complementary to an adjacent oligonucleotide block. In some embodiments, a modular barcode library may be formed by mixing two or more oligonucleotide blocks. In some embodiments, a modular barcode library may be formed by mixing equimolar amounts of oligonucleotide blocks. In some embodiments, a modular barcode library may be formed by mixing four oligonucleotide blocks. In some embodiments, a modular barcode library may be formed by subjecting the mixture of oligonucleotide blocks to heating, annealing, and/or ligation. One of skill in the art can appreciate that the parameters (e.g., duration, temperature, etc) of heating, annealing, and/or ligation depends on the nature and quantity of the oligonucleotide blocks used herein and can require optimization.

In some embodiments, a modular barcode library herein may comprise at least about 1 bp, at least about 2 bp, at least about 4 bp, or at least about 8 bp. In some embodiments, a modular barcode library herein may comprise about 1 bp to about 500 bp, about 2 bp to about 400 bp, or about 5 bp to about 300 bp. In some embodiments, a modular barcode library herein may comprise about 1 bp to about 300 bps, about 2 bp to about 275 bps, about 3 bp to about 250 bps, about 4 bp to about 225 bps, about 5 bp to about 200 bps, about 6 bp to about 175 bps, about 7 bp to about 150 bps, or about 8 bp to about 125 bps.

In some embodiments, a modular barcode library herein may comprise at least about 10 unique barcode sequences, at least about 25 unique barcode sequences, or at least about 50 unique barcode sequences. In some embodiments, a modular barcode library herein may comprise about 1 to about 5000 unique barcode sequences, about 15 to about 4000 unique barcode sequences, about 10 to about 3000 unique barcode sequences, about 20 to about 2000 unique barcode sequences, about 30 to about 2000 unique barcode sequences, about 40 to about 1000 unique barcode sequences, or about 50 to about 500 unique barcode sequences.

In some embodiments, a guest molecule herein can be recovered from the engineered host porous protein crystal. In some embodiments, a guest DNA herein can be recovered from the engineered host porous protein crystal after incubating the crystal in a mixture comprising dNTPs, ATP, or a combination thereof. In some embodiments, the incubation period may be about 1 minute, about 5 minutes, about 10 minutes, about 20 minutes, about 30 minutes, about 40 minutes, about 50 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8.5 hours, about 9 hours, about 9.5 hours, about 10 hours, about 10.5 hours, about 11 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 28.5 hours, about 29 hours, about 29.5 hours, about 30 hours, about 30.5 hours, about 31 hours, about 31.5 hours, about 32 hours, about 32.5 hours, about 33 hours, about 33.5 hours, about 34 hours, about 34.5 hours, about 35 hours, about 35.5 hours, about 36 hours, about 36.5 hours, about 37 hours, about 37.5 hours, about 38 hours, about 38.5 hours, about 39 hours, about 39.5 hours, about 40 hours, about 40.5 hours, about 41 hours, about 41.5 hour, about 42 hours, about 42.5 hours, about 43 hours, about 43.5 hours, about 44 hours, about 44.5 hours, about 45 hours, about 45.5 hours, about 46 hours, about 46.5 hours, about 47 hours, about 47.5 hours, or about 48 hours. In some embodiments, the amount of guest DNA incubated with a mixture comprising dNTPs, ATP, or a combination thereof may and will depend on the identity of the porous protein crystal and the at least one guest molecule. In some embodiments, the guest DNA herein recovered from the engineered host porous protein crystal comprises at least one synthetic barcode sequence. In some embodiments, the guest DNA herein recovered from the engineered host porous protein crystal may be subjected to any suitable method known in the art to read the least one synthetic barcode sequence encoded in the guest DNA. In some embodiments, the guest DNA herein recovered from the engineered host porous protein crystal may be subjected to PCR, qPCR, ddPCR, rtPCR, next-generation sequencing, or any combination thereof to detect the information encoded in the least one synthetic barcode sequence of the guest DNA. In some embodiments, guest DNA may be recovered from the crosslinked porous protein crystal with less than about 10% degradation, less than about 25% degradation, less than about 50% degradation, or less than about 75% degradation. In some embodiments, guest DNA may be recovered from the crosslinked porous protein crystal with about 1% to about 10% degradation, about 1% to about 25% degradation, about 1% to about 50% degradation, or about 1% to about 75% degradation. In some embodiments, guest DNA may be recovered from the crosslinked porous protein crystal with about 1%, about 2%, about 3%, about 4%, about 5%, about 6%, about 7%, about 8%, about 9%, or about 10% degradation. In some embodiments, guest DNA may be recovered from the crosslinked porous protein crystal without degradation.

In some embodiments, the at least one guest information storage medium herein may comprise a synthetic library. In accordance with these embodiments, a synthetic library may comprise at least one modular barcode library. In some embodiments, a synthetic library herein may comprise at least about one modular barcode library, at least about 5 modular barcode libraries, at least about 10 modular barcode libraries, at least about 25 modular barcode libraries, at least about 50 modular barcode libraries, at least about 75 modular barcode libraries, at least about 100 modular barcode libraries, at least about 125 modular barcode libraries, at least about 150 modular barcode libraries, at least about 175 modular barcode libraries, at least about 200 modular barcode libraries, at least about 225 modular barcode libraries, at least about 250 modular barcode libraries, at least about 275 modular barcode libraries, or at least about 300 modular barcode libraries. In some embodiments, a synthetic library herein may comprise about one modular barcode library to about 1000 modular barcode libraries, about 10 modular barcode libraries to about 900 modular barcode libraries, about 20 modular barcode libraries to about 800 modular barcode libraries, about 30 modular barcode libraries to about 700 modular barcode libraries, about 40 modular barcode libraries to about 600 modular barcode libraries, or about 50 modular barcode libraries to about 500 modular barcode libraries. In some embodiments, a synthetic library herein may be a synthetic next-generation sequencing (NGS) library. In some embodiments, modular barcode libraries may be modified in manner suitable for formation of a synthetic NGS library. In accordance with these embodiments, modular barcode libraries may be modified to include a Source Tag, a Trap Tag sequence, or a combination thereof.

In some embodiments, a synthetic library herein may comprise at least 100 reads of unique barcode DNA. In some embodiments, a synthetic library herein may comprise about 100 reads to about 500 million reads, about 500 reads to about 400 million reads, about 1000 reads to about 300 million reads, or about 1 million reads to about 200 million reads of unique barcode DNA. In some embodiments, a synthetic library herein may comprise about 10 million to about 200 million reads (e.g., about 10 million reads, about 15 million reads, about 20 million reads, about 25 million reads, about 30 million reads, about 35 million reads, about 40 million reads, about 45 million reads, about 50 million reads, about 55 million reads, about 60 million reads, about 65 million reads, about 70 million reads, about 75 million reads, about 80 million reads, about 85 million reads, about 90 million reads, about 95 million reads, about 100 million reads, about 110 million reads, about 120 million reads, about 130 million reads, about 140 million reads, about 150 million reads, about 160 million reads, about 170 million reads, about 180 million reads, about 190 million reads, about 200 million reads) of unique barcode DNA.

In some embodiments, a synthetic library herein may be recovered from the crosslinked porous protein crystal with less than about 10% degradation, less than about 25% degradation, less than about 50% degradation, or less than about 75% degradation. In some embodiments, a synthetic library herein may be recovered from the crosslinked porous protein crystal with about 1% to about 10% degradation, about 1% to about 25% degradation, about 1% to about 50% degradation, or about 1% to about 75% degradation. In some embodiments, a synthetic library herein may be recovered from the crosslinked porous protein crystal with about 1%, about 2%, about 3%, about 4%, about 5%, about 6%, about 7%, about 8%, about 9%, or about 10% degradation. In some embodiments, a synthetic library herein may be recovered from the crosslinked porous protein crystal without degradation.

### V. Methods of Making Data Storage Systems

In certain embodiments, the present disclosure provides methods of making the data storage systems disclosed herein. Some embodiments of the present disclosure encompass methods of making data storage systems herein wherein the methods may comprise preparing a porous protein crystal guest molecule conjugate. The method may comprise: (a) crystallizing a protein in appropriate crystal growth conditions to produce a porous protein crystal; (b) reacting the porous protein crystal with a crosslinking agent to produce a crosslinked porous protein crystal, wherein the crosslinking agent crosslinks adjacent monomers of the porous protein crystal; and (c) incubating the crosslinked porous protein crystal with at least one guest molecule to produce a porous protein crystal guest molecule conjugate.

In some embodiments, methods of making data storage systems herein may comprise preparing an engineered host porous protein crystal. In some embodiments, methods of making data storage systems herein may comprise crystallizing a protein to produce a porous protein crystal. In some embodiments, the protein may be crystallized according to the methods disclosed herein. In some embodiments, the crystallization protocol may make use of crystal seeds to enhance crystal growth. In some additional embodiments, the crystallization protocol may make use of crystal seeds that are stabilized by crosslinking according to methods disclosed herein.

In some embodiments, methods of making data storage systems herein may comprise crosslinking a porous protein crystal herein. In accordance with these embodiments, methods herein may comprise reacting the porous protein crystal with a crosslinking agent to produce a crosslinked porous protein crystal. In further embodiments, the crosslinking agent crosslinks adjacent monomers of the porous protein crystal. In some embodiments, the crosslinking agent may be as described herein.

In some embodiments, the present disclosure provides methods for preparing a porous protein crystal guest molecule conjugate for use in the data storage systems herein. The methods may comprise: obtaining a porous protein crystal, wherein the porous protein crystal has been reacted with a crosslinking agent to produce a crosslinked porous protein crystal and the crosslinking agent crosslinks adjacent monomers of the porous protein crystal; and incubating the crosslinked porous protein crystal with at least one guest molecule to produce a porous protein crystal guest molecule conjugate.

In some embodiments, methods of making data storage systems herein may comprise forming a porous protein crystal guest molecule conjugate. In accordance with these embodiments, methods herein may comprise incubating a crosslinked porous protein crystal with at least one guest molecule to produce a porous protein crystal guest molecule conjugate. In some embodiments, the at least one guest molecule may be any of those disclosed herein. In some embodiments, at least one guest molecule herein may be incubated with at least one porous protein crystal herein to produce a porous protein crystal guest molecule conjugate from about 1 minutes to about 48 hours. In some embodiments, the incubation period may be about 1 minute, about 5 minutes, about 10 minutes, about 20 minutes, about 30 minutes, about 40 minutes, about 50 minutes, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8.5 hours, about 9 hours, about 9.5 hours, about 10 hours, about 10.5 hours, about 11 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 24.5 hours, about 25 hours, about 25.5 hours, about 26 hours, about 26.5 hours, about 27 hours, about 28.5 hours, about 29 hours, about 29.5 hours, about 30 hours, about 30.5 hours, about 31 hours, about 31.5 hours, about 32 hours, about 32.5 hours, about 33 hours, about 33.5 hours, about 34 hours, about 34.5 hours, about 35 hours, about 35.5 hours, about 36 hours, about 36.5 hours, about 37 hours, about 37.5 hours, about 38 hours, about 38.5 hours, about 39 hours, about 39.5 hours, about 40 hours, about 40.5 hours, about 41 hours, about 41.5 hour, about 42 hours, about 42.5 hours, about 43 hours, about 43.5 hours, about 44 hours, about 44.5 hours, about 45 hours, about 45.5 hours, about 46 hours, about 46.5 hours, about 47 hours, about 47.5 hours, or about 48 hours. In some embodiments, the amount of the at least one guest molecule incubated with the at least one protein scaffold to produce a porous protein crystal guest molecule conjugate may and will depend on the identity of the porous protein crystal and the at least one guest molecule.

In some embodiments, the present disclosure provides methods for preparing a guest molecule for use in the data storage systems herein. In some embodiments, a guest molecule for use in the methods herein comprises at least one guest information storage medium.

In some embodiments, a guest molecule for use in the methods herein comprises at least one modular barcode library. In some embodiments, methods of generating modular barcode library may comprise constructing at least about two, at least about four, at least about six, at least about eight, at least about 10 oligonucleotide blocks from a pool of oligonucleotides disclosed herein. In some embodiments, methods of generating modular barcode library may comprise constructing about two to about 10, or about four to about eight oligonucleotide blocks from a pool of oligonucleotides disclosed herein. In some embodiments, methods of generating modular barcode library may comprise constructing about four oligonucleotide blocks from a pool of oligonucleotides disclosed herein. In some embodiments, oligonucleotide blocks are mixed together before subjecting the mixture to heating, followed by annealing, and then ligation. In some embodiments, about two to about 10 oligonucleotide blocks are mixed together before subjecting the mixture to heating, followed by annealing, and then ligation. In some embodiments, about four oligonucleotide blocks are mixed together before subjecting the mixture to heating, followed by annealing, and then ligation. Duration of heating, annealing, and ligation will depend on the oligonucleotide block mixture. Temperatures and temperature ranges at which heating, annealing, and ligation are performed will depend on the oligonucleotide block mixture.

In some embodiments, methods herein may further comprise reversing the binding of the guest molecule to the porous protein crystal. In some embodiments, guest molecules bound to the porous protein crystal may be released using acidic or basic solutions. In some embodiments, guest molecules bound to the porous protein crystal may be released from the porous protein crystal guest molecule conjugate using reducing conditions. In some embodiments, guest DNA bound to the porous protein crystal may be released by incubating the porous protein crystal guest molecule conjugate in a mixture of dNTPs, ATP, or any combination thereof. In some embodiments, guest DNA released from the porous protein crystal guest molecule conjugate may be recovered using PCR, qPCR, next-generation sequencing, or any combination thereof.

### VI. Methods of Use

In some embodiments, the present disclosure provides methods of use for data storage systems disclosed herein. In some embodiments, data storage systems disclosed herein may be used as a tracking system for at least one organism. For example, the crosslinked porous protein crystal comprising a synthetic library as presently disclosed herein may mark the at least one organism. Non-limiting examples of organisms suitable for the methods herein can include algae, bacteria, plants, insects, fish, amphibians, reptiles, birds, and mammals.

In some embodiments, data storage systems disclosed herein may be used as a tracking system for insects. In some embodiments, data storage systems disclosed herein may be used as a tracking system for Mosquitoes (family Culicidae); Horse flies and deer flies (family Tabanidae); Stable flies, house flies, and horn flies (family Muscidae); Sand flies (family Psychodidae); Black flies (family Simuliidae); Biting midges (family Ceratopogonidae); Bees, wasps, ants (order Hymenoptera); Butterflies and moths (order Lepidoptera); Beetles (order Coleoptera); Grasshoppers and katydids (order Orthoptera); True bugs (orders Hemiptera and Homoptera); Ticks (families Ixodidae and Argasidae); and the like. In some embodiments, data storage systems disclosed herein may be used as a tracking system for an organism that has been infected by an insect. In some embodiments, data storage systems disclosed herein may be used as a tracking system for an organism that has been bitten by an insect.

In some embodiments, data storage systems herein may be used as a tracking system wherein the organism ingests a crosslinked porous protein crystal comprising a synthetic library herein. In some embodiments, organisms may be fed a crosslinked porous protein crystal comprising a synthetic library herein ad libitum. In some embodiments, an insect may ingest a crosslinked porous protein crystal comprising a synthetic library herein. In some embodiments, an insect may ingest a crosslinked porous protein crystal comprising a synthetic library herein when the insect can be a larva, pupa, adult, or any combination thereof.

In some embodiments, data storage systems disclosed herein may be used as a tracking system for an organism that has been infected by an insect wherein the organism may be a mammal. In some embodiments, data storage systems disclosed herein may be used as a tracking system for an organism that has been infected by an insect wherein the organism may be a companion animal, such as but not limited to a cat, a dog, and the like. In some embodiments, data storage systems disclosed herein may be used as a tracking system for an organism that has been infected by an insect wherein the organism may be livestock, such as but not limited to a cow, a horse, a mule, a pig, a camel, a goat, and the like. In some embodiments, data storage systems disclosed herein may be used as a tracking system for an organism that has been infected by an insect wherein the organism may be a human.

### VII. Kits

In some embodiments, the present disclosure also provides kits for binding at least one guest molecule to a porous crystal protein. A kit may comprise, for example, a porous protein crystal that has been stabilized. The porous protein crystal may have a plurality of crystal pores with an average diameter of from about 3 nm to about 50 nm. The kit may further comprise a guest molecule. In other embodiments, the kit may further comprise materials and/or reagents for synthesizing a guest molecule herein. In other embodiments, the kit may further comprise materials and/or reagents for modifying a guest molecule so that it binds to the porous protein crystal. The kit may further comprise additional materials and/or reagents for incubating a guest molecule with the porous crystal protein. The kit may further comprise additional materials and/or reagents for reversing the binding of the guest molecule to the porous protein crystal.

In some embodiments, kits herein may comprise one or more buffers, oligonucleotides, and the like for use in preparing any of the data systems disclosed herein. In some embodiments, kits herein may comprise one or more materials and/or reagents for preparing barcode sequences as disclosed herein. In some embodiments, kits herein may comprise one or more materials and/or reagents for preparing oligonucleotide blocks as disclosed herein. In some embodiments, kits may comprise one or more materials and/or reagents for preparing synthetic libraries (e.g., NGS libraries) comprising barcode sequences as disclosed herein.

In accordance with these embodiments, kits may also provide a mixture of dNTPs, ATP, or any combination thereof for release of guest molecule (e.g., guest DNA, synthetic libraries, synthetic NGS libraries) from the porous crystal proteins herein.

In some embodiments, kits herein may comprise at one or more materials and/or reagents for applying the tracking systems herein to the organism of interest. In some embodiments, kits herein may comprise at one or more materials and/or reagents for using tracking systems herein after applying to the organism of interest.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. CJ Crystal Production

In accordance with the present disclosure, Campylobacter jejuni (CJ) protein crystals were prepared. Porous materials, such as zeolites, are essential components within mass transport-mediated industrial processes, including catalysis, separation and adsorption. Possessing a unique, periodic topology, known with atomic resolution, porous protein crystals presented as novel candidate materials for further study of the present disclosure.

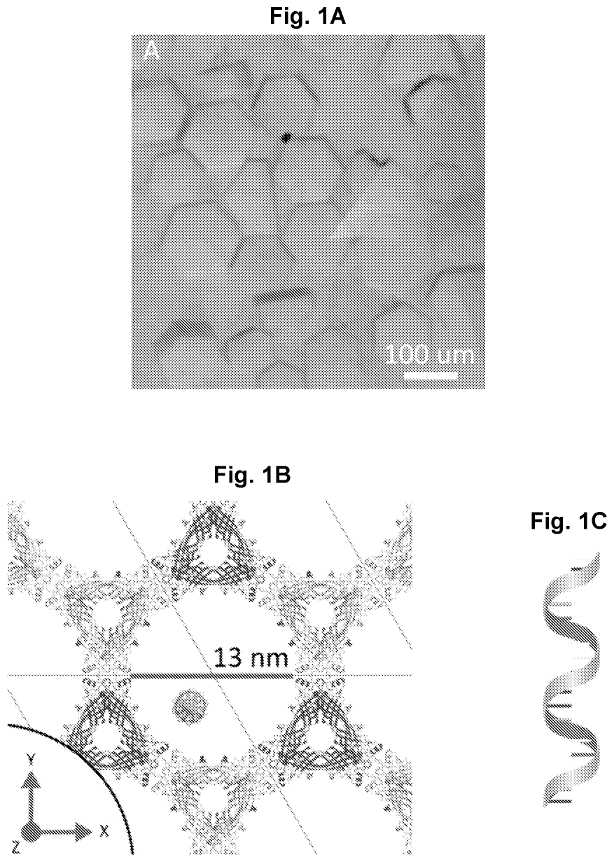

The porous protein crystals used in the exemplary methods herein were composed of a modified, periplasmic isoprenoid-binding protein derived from Campylobacter jejuni (Protein Data Bank ID: 2fgs), referred to as CJ, shown in FIGS. 1A-1B. In brief, CJ protein crystals were prepared using a CJ variant containing a C-terminal hexahistidine tag, CJ-6×HIS in pSB3 vector. The vector was expressed in E. Coli BL21 (Lucigen), grown in Terrific broth, followed by 0.4 mM IPTG induction for 16 hours at 25° C. Cells were sonicated into lysis buffer (50 mM HEPES, 500 mM NaCl, 10% glycerol, 25 mM imidazole, pH 7.4). Following centrifugation, lysate was purified by immobilized metal affinity chromatography (IMAC), specifically with a nickel bead-bound column (ThermoFisher Scientific HisPur Ni-NTA Resin). Following a single chromatography step, CJ-6×HIS was dialyzed into ammonium sulfate storage buffer (10 mM HEPES, 500 mM (NH4)2SO4, 10% glycerol, pH 7.4) overnight at 4° C. Purified protein was concentrated by ultracentrifugation (EMD Millipore) to 15 mg/mL, aliquoted, and stored at −80° C. Crystals were grown overnight by sitting drop vapor diffusion at 20° C. in 3.30-3.55 M (NH4)2SO4, 0.1 M Bis-tris at pH 6.0-6.5.

Crystal cross-linking was performed by first transferring crystals to a wash solution (4.2 M trimethylamine N-oxide (TMAO), pH 7.5) for approximately 1 hour to remove excess protein. Then, crystals were transferred to the cross-linking solution containing 40 mg/mL 1-ethyl-3-(3-dimethylaminopropyl) carbodiimide hydrochloride and 50 mM imidazole in 4.2 M TMAO, pH 7.5 in a covered well for 2 hours. Crystals were next transferred to a 50 mM borate buffer, pH 10 quenching solution for 1 hour. Crystals were washed and stored in 4.2 M TMAO, pH 7.5. As shown in FIG. 1B, CJ crystals contained a hexagonal array of 13-nm diameter axial pores, with lateral pores smaller than 3-nm in diameter.

### Example 2. DNA Loading of CJ Crystals

In accordance with the present disclosure, DNA was loaded into the CJ crystals prepared as described herein. Intra-crystal guest loading occurred primarily through the axial pores, allowing for adequate representation using a modified one-dimensional pore model (FIGS. 1C-1D). In brief, a single CJ crystal was placed in a polymethyl methacrylate (PMMA) microwell containing 2 μL of 0.4% low-melting point agarose and 3 μL of TE buffer, pH 7.4. The crystal, oriented on its side, was initially imaged under differential interference contrast and 488 nm light, at multiple planes along the z-axis. Following initial imaging, 5 μL of 50 μM FAM labeled single-stranded 8mer was added to the microwell and then sealed with tape. The crystal was then imaged at varying timepoints to capture guest loading. Loading experiments were performed in triplicate on a Nikon Eclipse Ti confocal microscope equipped with an Andor iXon Ultra 897U EMCCD camera. This protocol was followed for imaging loading with a FAM labeled double-stranded 15 bp guest. Both guest types were obtained from Integrated DNA Technologies.

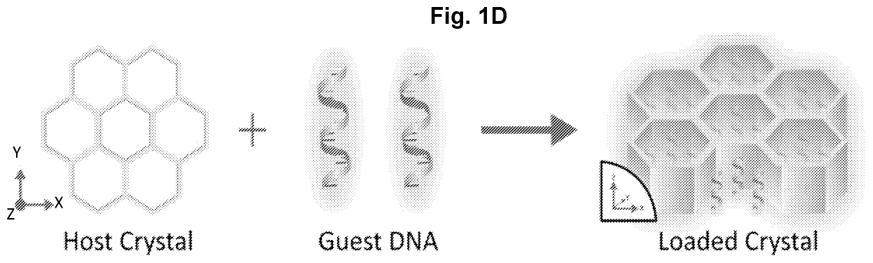

Separately a CJ crystal was placed in 100 μL of 1 ng/μL FAM labeled 125mer (5′-AGGCGACTCGACGGTCTTACGCGTTACGTATGATATGCATCACCACC ATCACCAATAACCAACACCTAAATTTAACATCCGAGAATTATGGAGCACGCTAGCG TACGCTACGGTCCTAACGCGC-3′ SEQ ID NO: 1) in TE, pH 8.0 and sealed overnight in a glass well plate. The crystal was then transferred to 100 μL fresh TE buffer to remove unbound guest for 1 hour. Following washing, the loaded crystal was transferred to 50 μL TE buffer on a glass slide and imaged under 488 nm light as described above to detect guest adsorption (FIG. 2A).

The confocal loading dataset for the FAM labeled 8mer single-stranded DNA guest (FIG. 2B) showed a crystal that was initially non-fluorescent. After the addition of the 8mer guest, a rapid increase in fluorescence was observed at the crystal sides, and the signal gradually increases towards the middle of the crystal. These results demonstrated that intra-crystal guest transport was mediated by the axial nanopores. Similar results were obtained for the FAM labeled 15mer double-stranded guest (FIG. 2C), but crystal loading occurred at a slower rate. The difference in relative loading rates between the two guest types suggested that guest size, and resultant rates of diffusion, influence the rate of guest adsorption, with guest of small mass diffusing into the crystals faster than guests with larger mass.

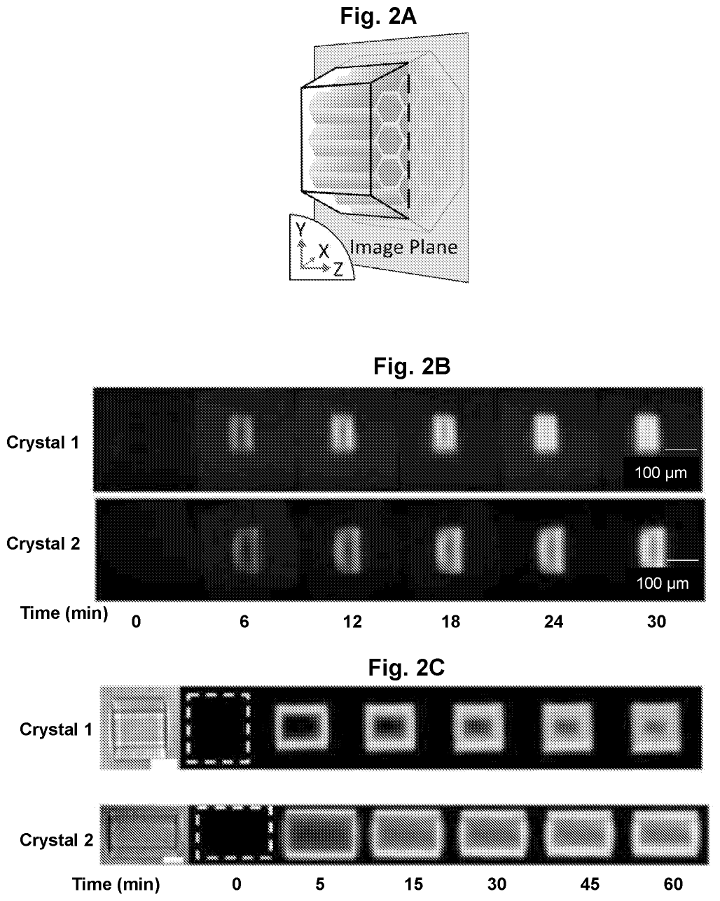

Confocal images of the 125mer loaded crystal (FIGS. 3A-3B) revealed localized areas of fluorescently bound guest. However, the relative amount of bound guest was much lower than the amount of 8mer and 15mer previously detected. The reduced 125mer guest amount bound to the host crystal was attributed to the difference in guest incubation concentrations, 25 μM for both 8mer and 15mer, and approximately 13 nM for the 125mer guest. Ideally, all guests would be loaded at the same concentration to allow for accurate comparison of experimental observation among all datasets. However, the difference in loading concentration was attributed to the manner in which the guests were obtained. Both 8mer and 15mer were purchased as 100 μM stocks from IDT. However, the 125mer guest was obtained as a 250 ng gBlock from IDT and then PCR amplified, thus limiting the amount of guest available for experimental trials, resulting in a reduced loading concentration and a reduced adsorbed guest amount.

### Example 3. Crystal Adsorbed DNA Recovery

In accordance with the present disclosure, the crystal adsorbed DNA was recovered with PCR. In brief, A CJ crystal was placed in 100 μL of 1 ng/μL 125mer (SEQ ID NO: 1) in TE, pH 8.0 and sealed in a glass well overnight. Following guest loading, the crystal was washed in 100 μL of TE to remove unbound 125mer in solution. The crystal was then transferred to 100 μL of a 10 mM dNTP mixture and sealed in a glass well overnight to trigger release of crystal-adsorbed 125mer into the surrounding solution. Following dNTP incubation, 1 μL of the solution was used as the template for PCR to test for recovery of crystal-adsorbed guest with the following primers: Forward: 5′-TAGGCGACTCGACGGTCTTACGCGTTACGT-3′(SEQ ID NO: 2); Reverse: 5′-GCGCGTTAGGACCGTAGCGTACGCTAGCGT-3′ (SEQ ID NO: 3). Reaction conditions were: 1 cycle of 95° C. for 3.00 minutes and 40 cycles of 95° C. for 15 seconds followed by 72° C. for 30 seconds. PCR was performed using Q5 High-Fidelity Polymerase (New England Biolabs). Following PCR, the products were examined for the 125mer amplified template using agarose gel electrophoresis.

Agarose gel electrophoresis was used to assess recovery of previously crystal bound 125mer guest. As shown in the gel image (FIG. 3C), the 125mer product was successfully amplified following dNTP triggered release. Recovery of crystal bound DNA using PCR demonstrated that the guest sequence remained accessible despite storage within host crystals. Furthermore, dNTP-triggered release of guest DNA suggested that the dNTPs at high concentration out-competed guest DNA for binding sites along the crystal nanopores, displacing 125mer guest into the surrounding solution that was recovered with PCR.

### Example 4. Validation on ATP Flushed Crystals

In accordance with the present disclosure, ATP flushed crystals were validated in vitro using qPCR. To prepare crystals for qPCR, 4 CJ crystals (200 μm diameter) were immersed in 18 μL of approximately 30 ng/μL of a 125 base pair double-stranded DNA oligonucleotide (125mer, SEQ ID NO: 1) and sealed in a glass well plate for approximately 12 hours, followed by washing with loading buffer (10 mM HEPES, 50 mM MgCl2, 10% glycerol, pH 8.0) to remove unbound 125mer. Crystals were then immersed in 18 μL of 20 mM ATP and an aliquot of the solution was stored. The 125mer loaded crystal/ATP solution was sealed in a glass well plate for approximately 12 hours. Following the 12-hour incubation, an additional aliquot of the solution was stored to compare solution 125mer concentration pre- and post-ATP incubation via qPCR with primers having the sequences of SEQ ID NOs: 2 and 3.

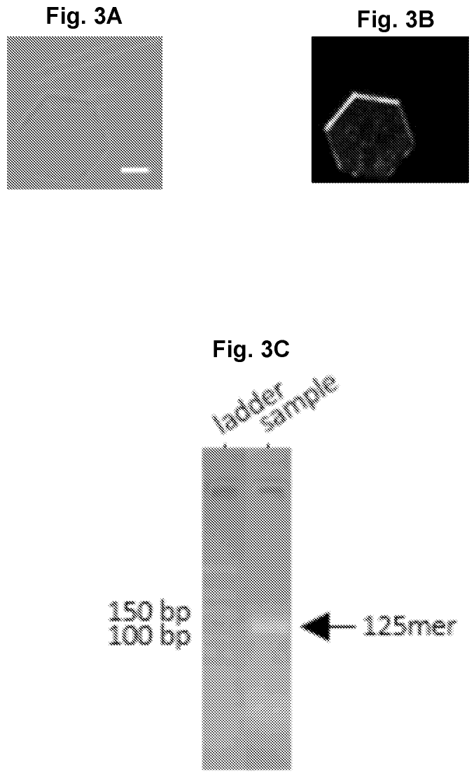

To confirm ATP incubation is promoting crystal release of guest DNA, qPCR was performed on samples of the solution DNA pre- and post-ATP incubation. The results (FIG. 4) showed that before ATP incubation, the 125mer was detected, although at nearly 3 orders of magnitude less than the amount in the smallest standard. Following 12-hour ATP incubation, the solution guest amount increased by several orders of magnitude, within the range of the standard curve. These results confirmed that ATP incubation of crystals loaded with guest DNA, triggered the release of bound guest into the surrounding solution, allowing the guest to be recovered by PCR and qPCR.

In sum, the present disclosure demonstrated a greater characterization of guest DNA adsorption-coupled diffusion within host porous protein crystals. The loading of guest DNA occurred primarily along the axial nanopores and the rate of intra-crystal guest diffusion changed overtime due to transport hindrances created from adsorbed guest molecules. Examples herein showed that a mixture of dNTPs, or ATP, could be used to trigger the release of crystal-adsorbed DNA, allowing for recovery using PCR or qPCR. Given the robust crystal scaffold following cross-linking, applications may include, but are not limited to, employing protein crystals to house and protect DNA laden with information as a novel tracking material (e.g., a DNA barcode library).

### Example 5. Constructing a DNA Barcode Library

The growing demand for data storage is expected to surpass the world's estimated silicone supply with the next few decades. Several inherent properties of DNA contribute to it serving as an information storage medium including high encoded information density, stability and virtually guaranteed access to the requisite machinery for writing and reading DNA.

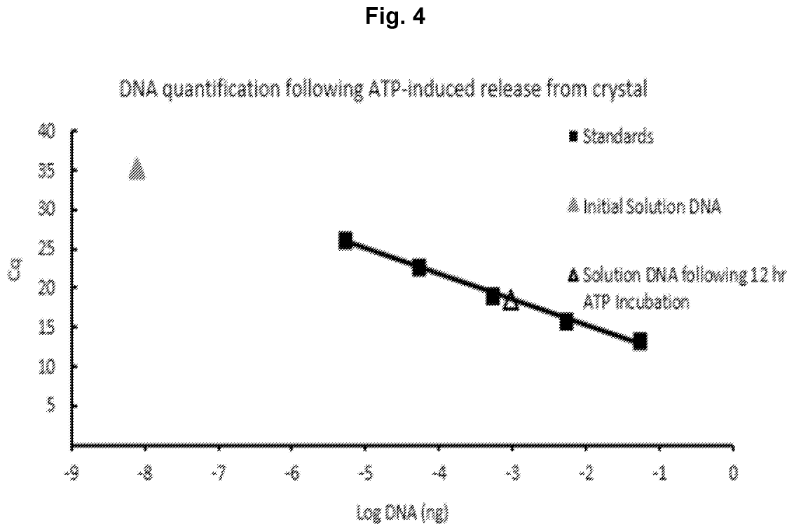

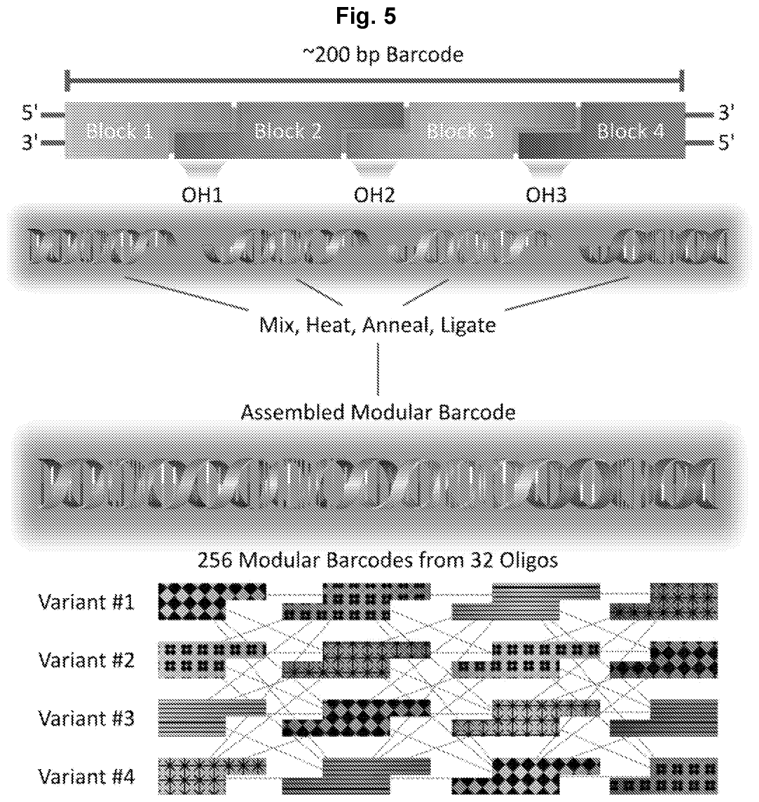

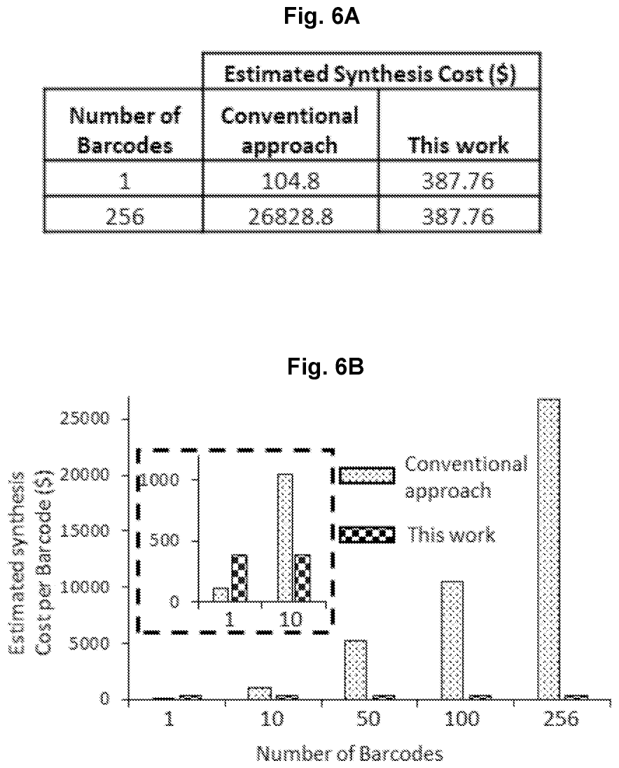

The goal of the present disclosure was aimed, not only at encoding arbitrary data in DNA, but to also push the limit of economic DNA barcoding by maximizing the number of DNA barcodes (arbitrary groups of unique DNA sequences) possible for marking objects/organisms while minimizing synthesis and sequencing costs. Herein, the present disclosure demonstrated (1) construction of a modular DNA library comprising interchangeable ‘blocks’ (FIG. 5) with multiple variants for increasing the number of barcode sequences possible from a handful of oligonucleotides; (2) recovery of 100s of unique barcode sequences from only 32 oligonucleotides highlighting the economic (FIGS. 6A-6B) and scalable benefits of this novel DNA barcode approach; and (3) powercode design (FIG. 7).

FIG. 5 shows a schematic of modular barcode design and assembly. In brief, the 131 bp modular barcode was constructed from 4 oligonucleotide ‘blocks’ containing single-stranded DNA overhangs complementary to neighboring blocks. The sequence specificity of each barcode was achieved by the unique nucleotide sequence within each block. Barcode assembly involved mixing equimolar amounts of each block together, followed by heating, annealing, and ligation. Since 4 sequence variants of each block were obtained, this allowed for assembly of 256 unique modular barcodes from 32 oligonucleotides.

FIG. 6A shows a table comparing estimated synthesis cost for 1 and 256 barcodes, separately, between the conventional approach and the approaches disclosed herein. FIG. 6B shows a comparative bar chart of increasing estimated oligo synthesis cost per the conventional approach of purchasing pre-made oligos of the desired length. However, the novel approaches disclosed herein allowed for assembly of up to 256 barcodes for less than 1.5% of the cost. The graph inset of FIG. 6B highlights cost difference at low number desired barcodes.

FIG. 7 shows an exemplary example of powercode design according to methods of the present disclosure. In brief, from a pool of 256 modular dsDNA barcodes, powercodes were created by combining multiple barcode strands into a single sample. The number of barcodes combined, k, determined the number of possible powercodes that could be created from the 256 modular barcodes (see top of FIG. 7). Each block depicted in the middle of FIG. 7 represents a unique barcode strand that was designated with an index value. Shaded blocks represent the specific dsDNA barcodes included within a particular powercode subset. As the number of barcodes per powercode increased, the available pool of samples containing a unique combination of sequences greatly exceeded the initial 256 modular barcode pool constructed with only 32 oligonucleotides (see bottom of FIG. 7).

### Example 6. Marking Organisms with Unique Barcodes

West Nile Virus (WNV) is a mosquito-borne disease capable of causing severe illness. Increased surveillance into WNV-spreading mosquitoes, such as Culex tarsalis, can inform public health personnel on areas of high mosquito productivity and the dynamics of arbovirus circulation, allowing targeted control interventions.

Currently, one of the most standard and comprehensive tools entomologists use to measure these parameters in nature is mark-release-recapture (MRR) studies. By marking a subsample of mosquitoes in the environment and monitoring their recapture rates and distances from release site, MRR offers a standard approach to gathering this epidemiologically significant information on mosquito behavior and ecology directly from field populations. Despite their utility, mosquito MRR studies represent a research area that has posed significant challenges to entomologists for decades. For mosquito dispersal studies, topical fluorescent powders and paints, ingestible dyes, or larval habitat marking with rubidium, or stable isotopes, are typically used. Despite being the most popular and cheapest option, fluorescent powders are difficult to use for large numbers of mosquitoes, they have limited surface stability on the mosquito, and they can introduce biases by negatively affecting mosquito behavior and survivorship. Mosquitoes reared from larval habitats enriched in stable isotopes or rubidium can be detected via mass spectrometry or x-ray fluorescence spectrophotometry, respectively. Nevertheless, these methods only provide a handful of distinguishable markers, and detection via mass spectrometry is expensive and training intensive. To overcome these challenges, the present disclosure developed a new class of MRR markers based on synthetic DNA barcodes. Specifically, the present disclosure designed a synthetic next-generation sequencing (NGS) library encoded with information (barcode DNA) as an insect tracking approach. The library was stored and protected throughout tracking experiments within crosslinked porous protein crystals. Under the disclosed strategies, mosquito larvae were marked with unique barcodes upon ingestion of these microcrystals.

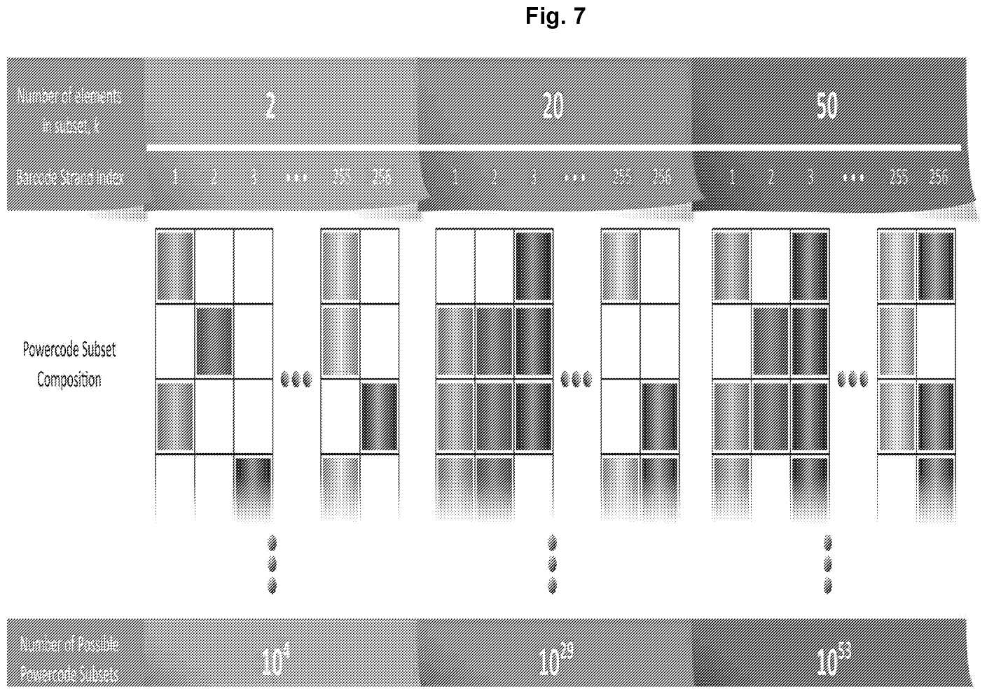

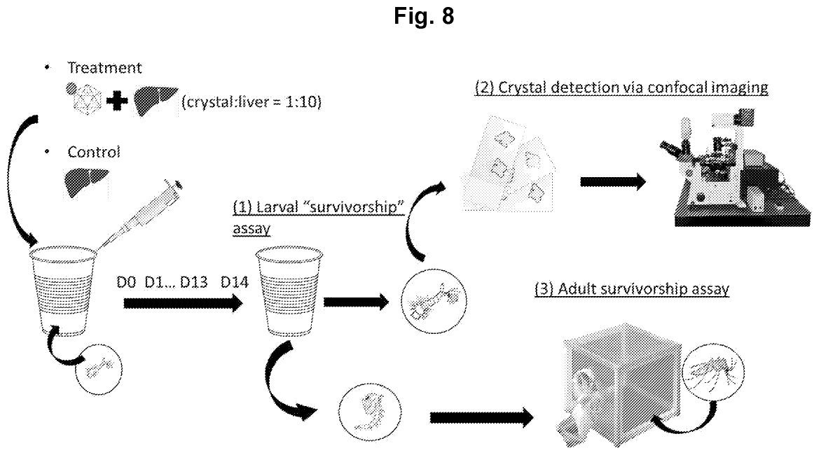

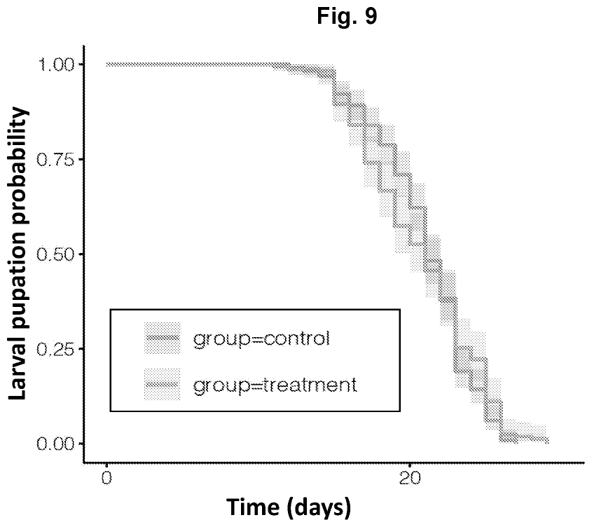

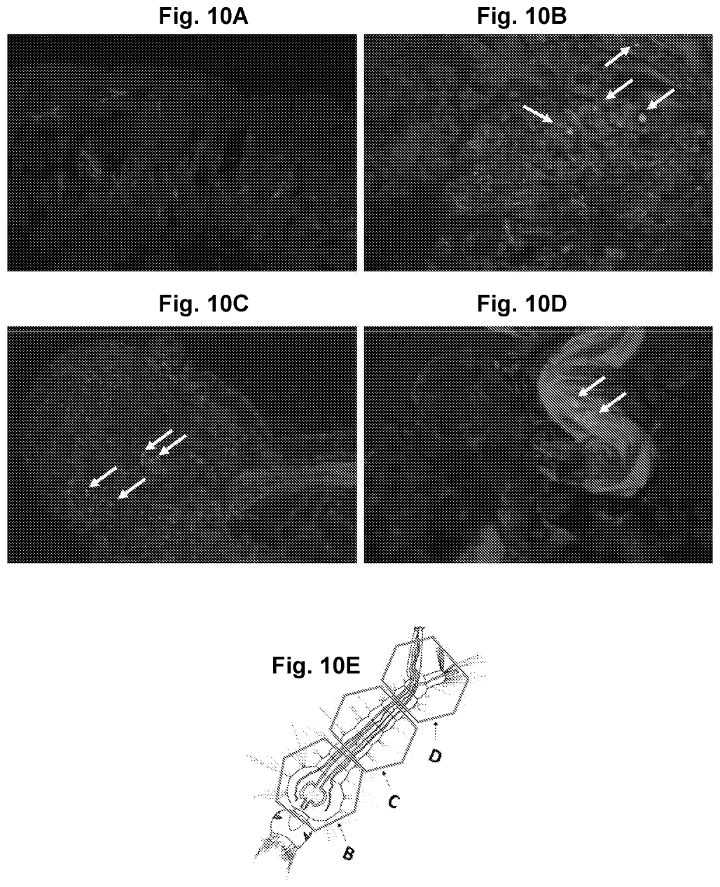

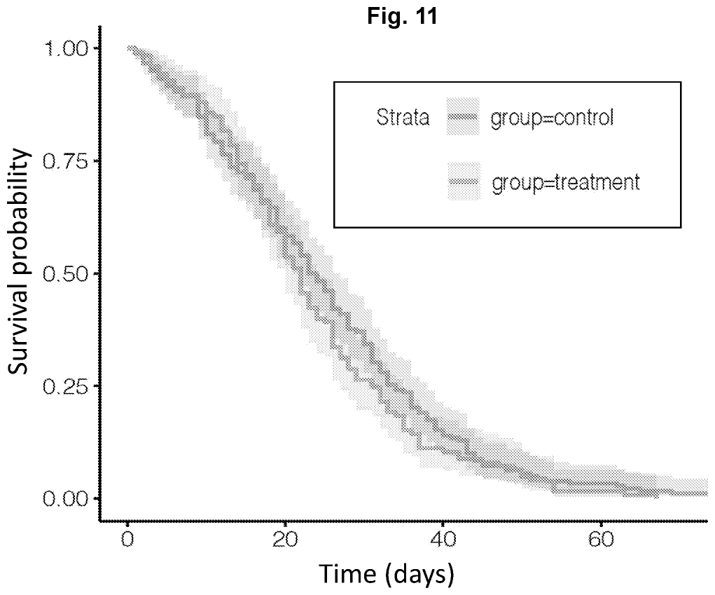

Hexagonal protein crystals composed of an isoprenoid binding protein derived from Campylobacter jejuni were prepared according the methods disclosed herein. The resulting protein crystals comprised of an array of nanopores that were 13 nm in diameter, allowing for inward diffusion and adsorption of DNA barcodes to nanopore walls. FIG. 8 shows an exemplary experimental design to test the viability of microcrystals as mosquito larvae markers. In brief, Cx. tarsalis first instar larvae were placed in 9 oz containers and received 1 of 2 diets during the duration of their larval development stages: Control: 10% liver powder only; Treatment: 10% microcrystals conjugated w/ Texas red fluorophores in 10% liver powder. First a larval survivorship assay was conducted to measure the number of larvae that successfully made it to the pupation stage. Data showed that ingestion of microcrystals did not affect survivorship and, as shown in FIG. 9, microcrystal ingestion had no impact on larval development. To determine if larvae ingest the microcrystals, larvae were subjected to confocal microscopy to detect the Texas red fluorophores conjugated to the microcrystals. FIGS. 10B-10D show markedly brighter fluorescence in treatment larvae compared to control-fed larvae (FIG. 10A) demonstrating the presence of crystals at different portions of the gastrointestinal tract (foregut (B), midgut (C), and hindgut (D) as indicated in FIG. 10E). Survival was also determined for adult Cx. tarsalis and it was found that microcrystal ingestion during larval development did not have downstream survival implications for mosquito adults (FIG. 11). Finally, to determine if microcrystals ingested during larval development could persist into adulthood, images were taken of the larvae gut (FIG. 12A) and the gut from dissected adult Cx. tarsalis (FIG. 12B). Fluorescence imaging showed Texas-red conjugated crystals in both the Larvae and the adult. These data demonstrated that DNA-loaded microcrystals were freely ingested by developing Cx. tarsalis mosquito larvae with no significant impact on larvae or adult survival rates and that the crystals persisted into adulthood.

To evaluate the performance of DNA barcode loaded protein crystals as a novel mosquito tracking material, crystal-loaded barcodes, ˜200 bp, were incubated for 24-hours with mosquito homogenate. To test for library recovery, samples of each solution post-incubation were used as the template for PCR and analyzed via agarose gel electrophoresis. As a field component to mimic realistic environmental conditions, mosquitoes were trapped at designated field sites containing water-filled tubs spiked with barcode loaded crystals. Barcode detection from collected field samples was performed using quantitative PCR (qPCR). Barcode positive field samples were prepared for NGS by 2 rounds of overhang PCR to append illumina adapters. Lastly, the modular barcodes were constructed out of smaller double-stranded ‘blocks’ containing single-stranded overhangs for annealing in the targeted linear order. The modular library was designed using python, scored with the nucleic acid secondary structure prediction program NUPACK (See Zadeh, et al., J Comput Chem, 32:170-173, 2011, the disclosure of which is incorporated herein in its entirety) and validated using NGS.

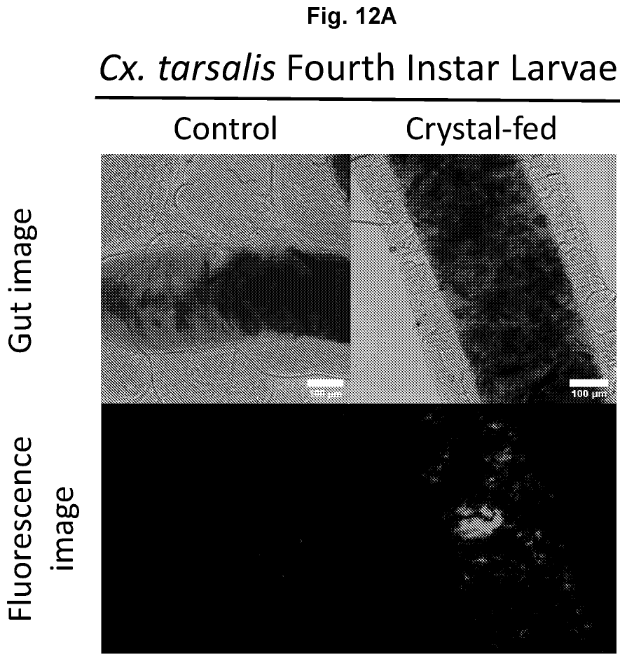

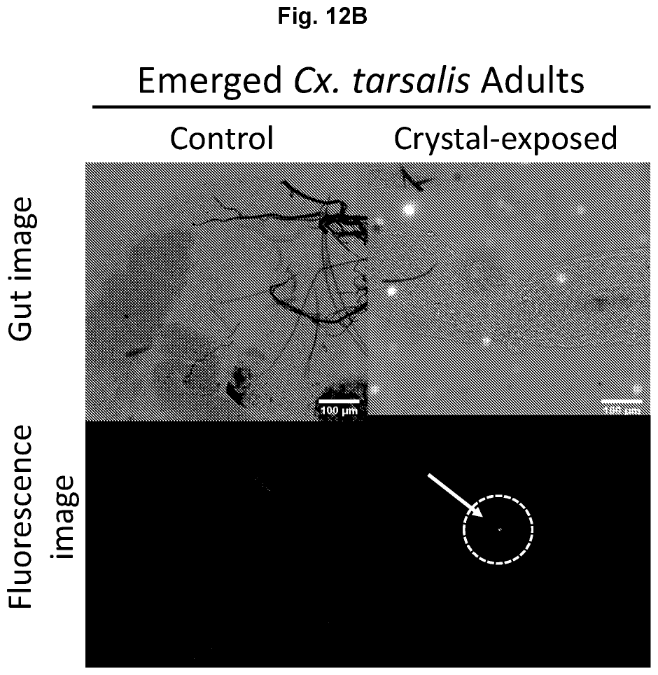

Barcode DNA in solution with mosquito homogenate was not recovered using PCR (FIG. 13). Remarkably, crystal-loaded barcode DNA mixed with mosquito homogenate was recovered by PCR (FIG. 13), suggesting that porous protein crystals serve as a robust material for storing and protecting barcode DNA. Barcode positive field samples detected via qPCR displayed additional peaks in the melt curve suggestive of possible non-barcode amplification (FIG. 14). However, NGS results showed numerous barcode fragments capable of contributing to additional peaks observed in qPCR melt curves (FIG. 14). The modular library NGS results consisted of approximately 37 million reads of the designed barcode demonstrating DNA barcodes can be assembled from smaller ‘blocks’ allowing for numerous distinct barcode generation by incorporating ‘block’ variants containing unique, internal sub-barcodes.

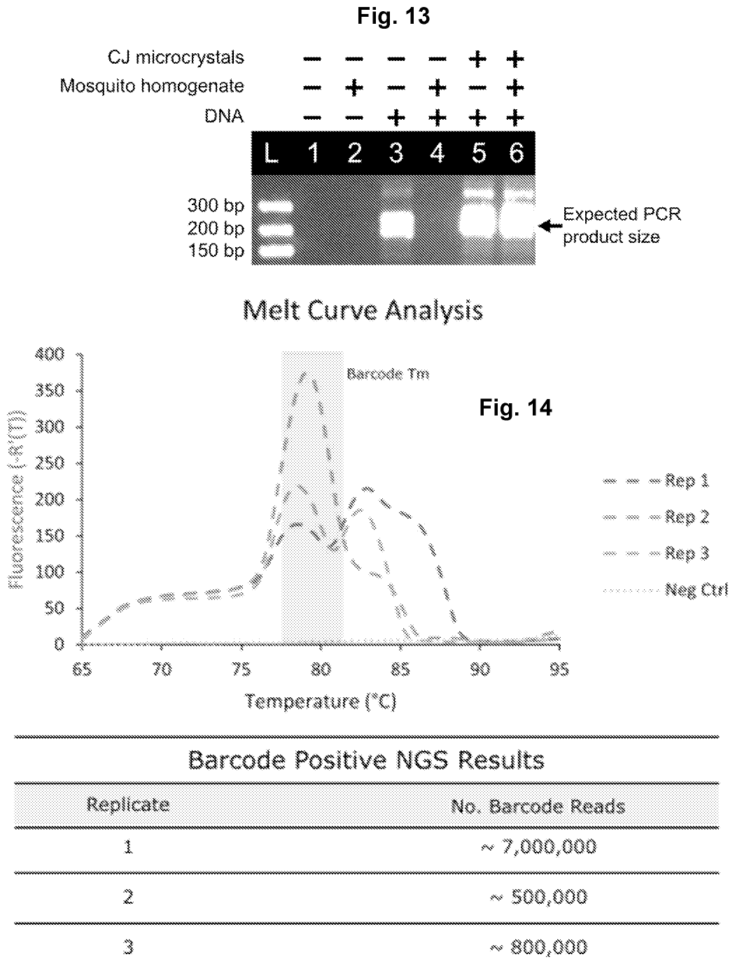

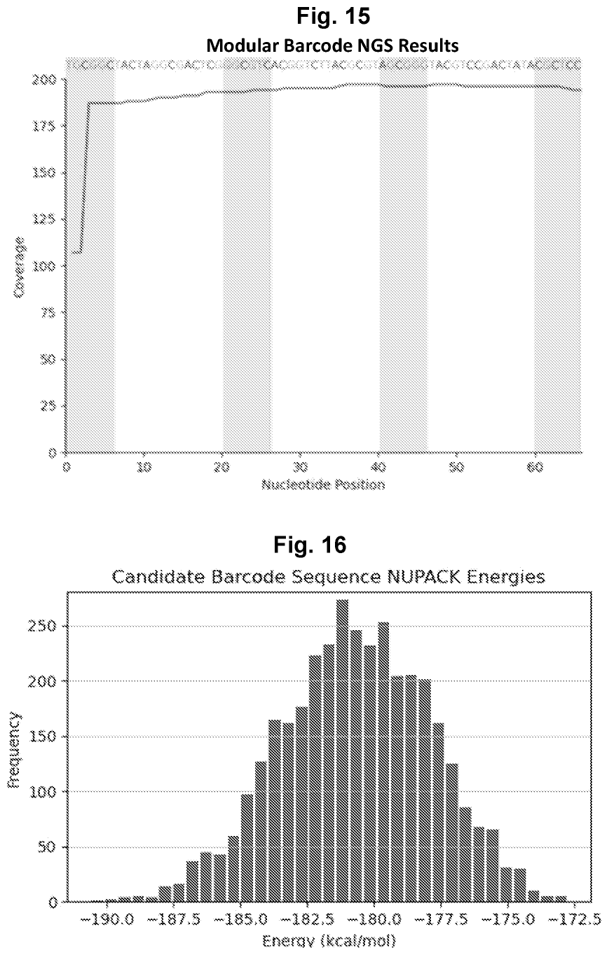

NGS coverage results for a single modular barcode, shown in FIG. 15, were determined using the software Geneious Prime for the top 100 reads which displayed markedly high coverage across the entire barcode sequence, including the variable 6nt regions shaded in gray. The histogram of NUPACK scoring results (FIG. 16) for each of the ˜3,600 candidates generated, revealed a bell-shaped distribution with an average of approx. −181 kcal/mol describing the propensity of each candidate, comprised of 8 single-stranded oligonucleotides, to form the target secondary structure. The top 4 scoring candidates were chosen for experimental validation.

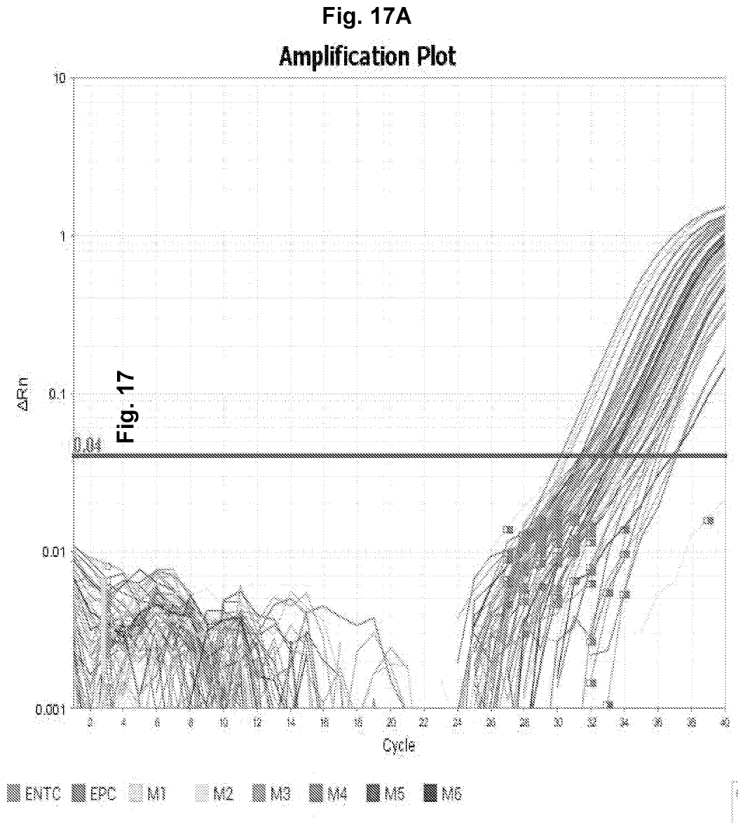

Barcode was amplified from adult male mosquitoes reared on (fed) barcode-laden microcrystals as larvae. qPCR amplification plots showed barcode amplification from mosquito samples between 24-32 cycles (FIG. 17A). Positive controls represented serial dilutions of naked barcode. Negative controls represent PCR master mix with no template added. Each well contained one homogenized male mosquito (FIG. 17B). These data provided additional evidence that barcodes can be recovered from adult mosquitoes that were fed barcode-containing crystals as larvae. Additionally, data demonstrated crystal prevalence in emerged male mosquitoes.

In sum, DNA barcode loaded protein crystals possessed an elevated resistance against degradation despite incubation in mosquito homogenate. DNA barcodes from loaded crystals previously ingested by mosquito larvae did not influence survival and were detectable using both qPCR and NGS. The modular barcode design and validation demonstrated that DNA barcodes can be assembled from smaller ‘blocks’ allowing for numerous distinct barcode generation by incorporating ‘block’ variants containing unique internal sequences. Importantly, the utilization of a modular barcode design and NGS platform for analysis permits the simultaneous detection of multiple barcodes from each field-collected mosquito pool, which is not a capability of current technologies.

### Example 7. Barcode Recovery and Validation

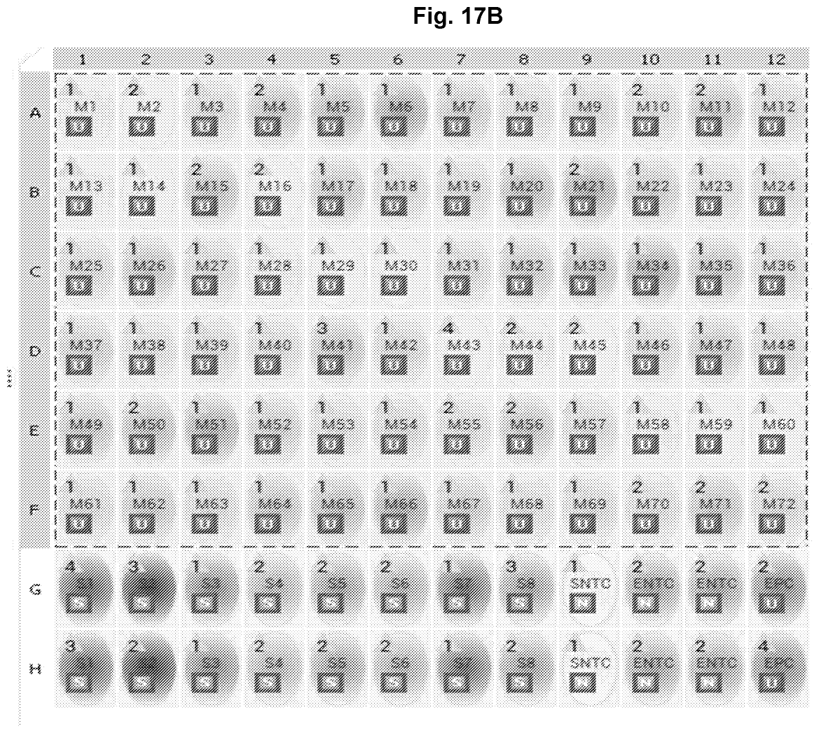

In accordance with the present disclosure, mosquito larvae were marked with unique barcodes upon ingestion of these microcrystals as disclosed herein. Mosquitoes that were exposed to CJ crystal loaded with a synthetic barcode sequence were then subjected to homogenization and DNA extraction. Two primers were selected to amplify an 84-nt segment of synthetic barcode in qPCR experiments with these samples. Samples included three mosquitoes that were reared on (fed) barcode-laden microcrystals as larvae and from which barcode was detected in the emerged adult mosquitoes (SR1-3), three pools of wild-caught mosquitoes that colonized a crystal-spiked tub in the field and were later captured as adults in a CDC light trap (FR1-3), and three wild-caught mosquitoes that colonized crystal-spiked tubs placed in the field as larvae and were reared to adults in the laboratory (LR1-3). The positive control was naked barcode. The negative control was PCR master mix with no template added. The qPCR melt curves (FIG. 18) had peaks corresponding to the target 84mer barcode peak in addition to a slightly higher peak (˜82-84° C.) from samples obtained from survivorship and field studies.

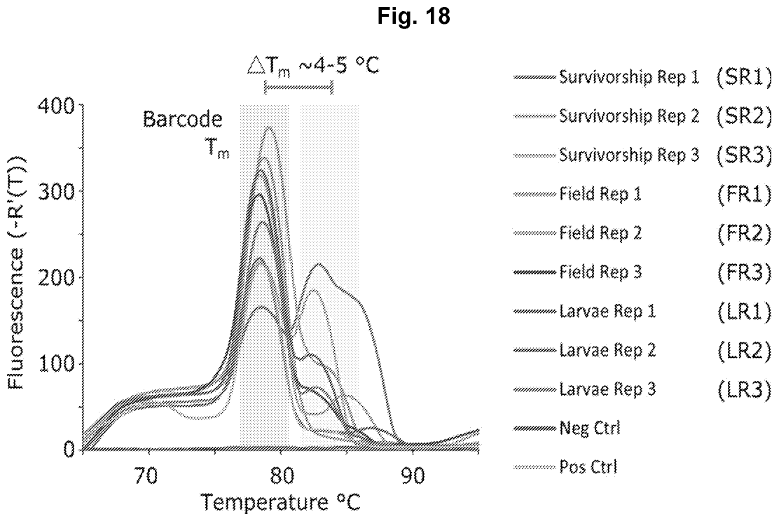

To further verify that the qPCR signal was coming from authentic barcode, we proceeded to check the size of the PCR amplicon using gel electrophoresis (FIG. 19). All the samples that were exposed to the synthetic barcode sequence had PCR amplified output products with the expected size for the 84-bp product. There was also a distinct and repeatable band for a somewhat larger product. A longer side product was consistent with the small peaks observed in qPCR with higher melting temperature.

To verify even further that the recovered samples contain authentic barcode sequences, and that the side product that contributes to the qPCR signal arises from the synthetic barcode, next-generation sequencing (NGS) was used. Specifically, the larger band was extracted from the electrophoresis gel, added flanking adaptors for NGS using additional rounds of PCR, and proceeded with NGS.

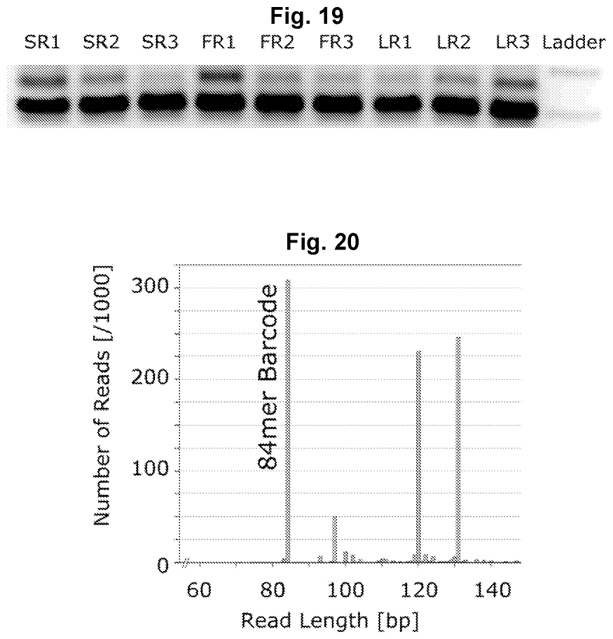

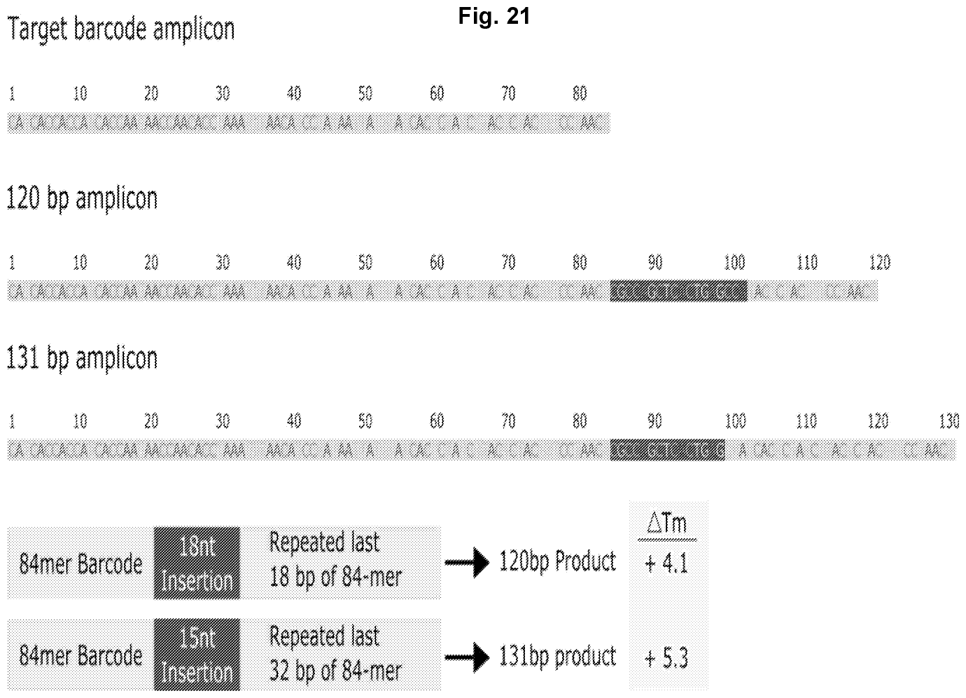

As shown in an analysis of 1 million aligned reads (FIG. 20), performed in Geneious Prime, the most common read corresponded to the expected 84-bp sequence, despite extracting the larger band size. The other two dominant read sequences corresponded to 120 bp and 130 bp sequences (FIG. 21) that are almost entirely composed of the original synthetic barcode, with insertions/duplications suggestive of an earlier off-target amplification event (e.g. mispriming) during the synthetic barcode production. Notably, estimated Tₘ for these two longer products (FIG. 21) were similar to the higher melt temperature region shown in FIG. 18.

### Example 8. Modular Barcodes Synthesis and Validation

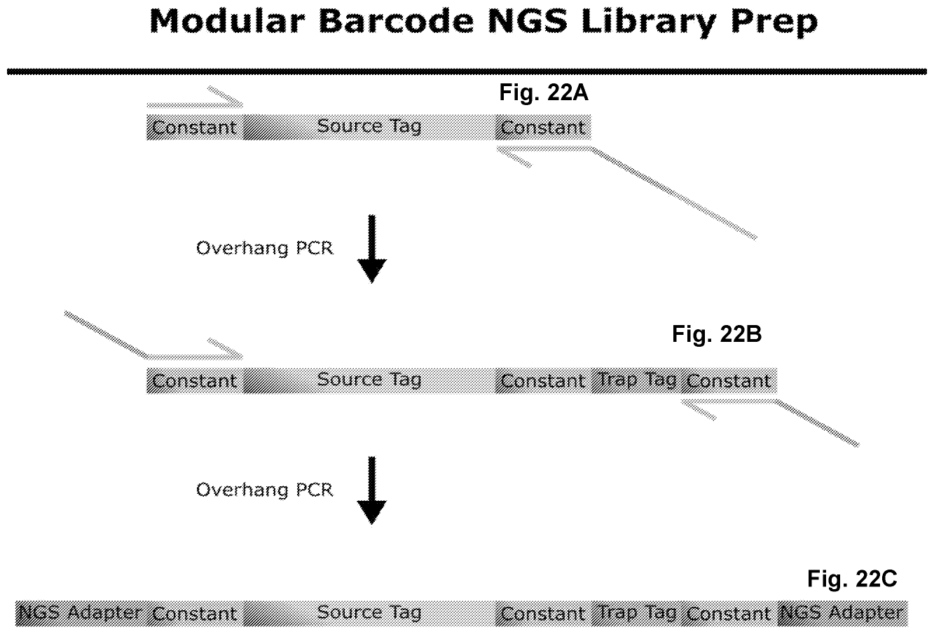

Modular barcodes were synthesized by mixing, annealing and ligating 8 single-stranded oligonucleotides to form the core barcode (FIG. 22A) comprised of the Source Tag region flanked by two regions of constant sequence shared among all modular barcode variants. Following Source Tag construction, several rounds of overhang PCR are performed to append the unique Trap Tag sequence (FIG. 22B), and the illumine adapter sequences necessary for Input to Next-Generation Sequencing (NGS) platforms (FIG. 22C).

Modular Barcodes were subjected to experimental validation. In brief, all 32 oligos corresponding to the 4 variants for each of the 4 blocks were purchased from Integrated DNA Technologies (Coralville, Iowa) with 6 oligos containing a 5′ phosphate. Each oligo was resuspended to a stock concentration of 100 μM in duplex buffer (100 mM Potassium Acetate, 30 mM HEPES, pH 7.5). A 0.02 pmol/μL working solution was made from each stock solution using duplex buffer. From each of the 8 working solutions corresponding to a single modular barcode sequence, 2 μL was transferred to a 0.2 mL PCR tube and mixed. The mixture was then heated to 94° C. for 4 minutes using a heat block, followed by gradual cooling for 1 hr by turning off the heat block. Following annealing, 2 μL T4 DNA Ligase Buffer (NEB) and 1 μL T4 DNA Ligase (NEB) were added to the annealed mixture followed by incubation at room temperature for 10 minutes. The ligation reaction was heat inactivated by 10-minute incubation at 65° C. The inactivate ligation mixture was used as the template for overhang PCR with the following reaction conditions: 1 cycle of 98° C. for 45 seconds, 30 cycles of 98° C. for 30 seconds, 61° C. for 30 seconds, 72° C. for 30 seconds, and 1 cycle of 72° C. for 1 minute. Overhang PCR was used with the following primer sequences: fwd 5′-ACACTCTTTCCCTACACGACGCTCTTCCGATCTCCAGTCCTCAACAAGCTG-3′ (SEQ ID NO: 4); rev 5′-GTTGAAGCCGGTTACCAC-3′ (SEQ ID NO: 5). Three additional rounds of overhang PCR were performed with the following primer sets: #1, fwd: 5′-ACACTCTTTCCCTACACGACGCTCTTCCGATCT-3′ (SEQ ID NO: 6); #1 rev 5′-TTCTGGGTTCCTCATCGCNNNNNNNNGTTGAAGCCGGTTACCAC-3′ (SEQ ID NO: 7); #2, fwd: 5′-ACACTCTTTCCCTACACGACGCTCTTCCGATCT-3′ (SEQ ID NO: 8); #2 rev: 5′-GTGACTGGAGTTCAGACGTGTGCTCTTCCGATCTTTCTGGGTTCCTCATCGC-3′ (SEQ ID NO: 9); #3, fwd: 5′-AATGATACGGCGACCACCGAGATCTACACTCTTTCCCTACACGACGCTCTTCCGAT CT-3′ (SEQ ID NO: 10); #3 rev: 5′-CAAGCAGAAGACGGCATACGAGATNNNNNNNNNNATATTCACGTGACTGGAGTTC AGACGTGTGCTCTTCCGATCT-3′ (SEQ ID NO: 11). All PCR was performed using the same thermocycling conditions as described above. Following amplification, PCR cleanup was performed using KAPA Pure Beads (Roche). Size selection for the 262 bp barcode library was performed using Monarch DNA Gel Extraction Kit (New England Biolabs). The library was quantified using Qubit lx dsDNA HS Assay Kit (ThermoFisher) and diluted to 20 nM for sequencing sample prep. Paired end 2×150 cycle sequencing was run on an illumina NovaSEQ 6000 (Genomics and Microarray Core, University of Colorado Anschutz Medical Campus). The ea-utils package was used for initial sample processing including adapter trimming and read joining. FastQC was used to check overall quality of joined reads and to determine total read count of detected barcode (Babraham Bioinformatics). Additional scripts written in python were used for subsequent analysis and visualization of barcode recovery in read data.

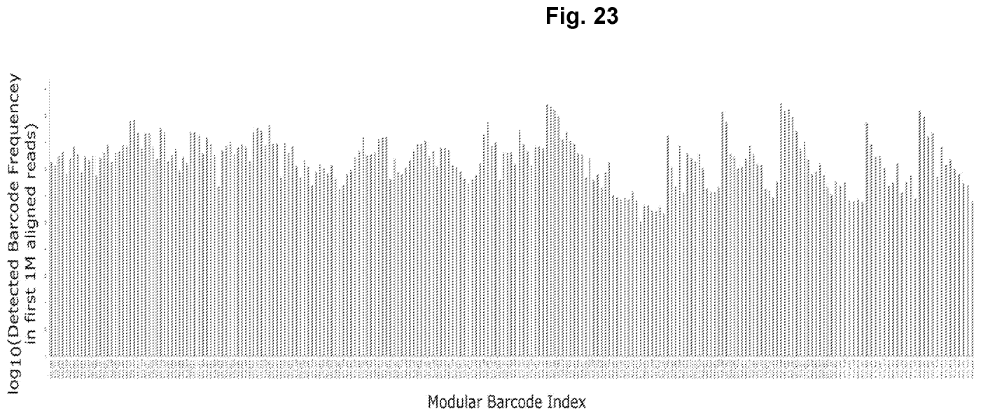

Nearly all 256 modular barcode variants (234) were detected simultaneously in the first 1 million aligned reads out of 80 million reads via NGS in relatively similar proportions (FIG. 23). The remaining 79 million reads could confirm detection of the remaining 22 variants following alignment, thus validating the modular barcode NGS-compatible library design.

1.-19. (canceled)

20. A tracking system for organisms, the tracking system comprising
a synthetic library encoded with unique barcode DNA sequences, and
a crosslinked porous protein crystal, wherein the synthetic library is stored in the crosslinked porous protein crystal.

21. The tracking system according to claim 20, wherein the synthetic library comprises a synthetic next-generation sequencing (NGS) library.

22. The tracking system according to claim 20, wherein the synthetic library comprises at least one modular barcode library.

23. The tracking system according to claim 20, wherein the at least one modular barcode library comprises at least four oligonucleotide blocks, wherein an oligonucleotide block comprises a DNA overhang complementary to an adjacent oligonucleotide block.

24. The tracking system according to claim 20, wherein the at least one modular DNA barcode comprises about 5 base pairs (bp) to about 300 bp.

25. The tracking system according to claim 20, wherein the at least one modular barcode library comprises about 50 to about 500 unique barcode sequences.

26. The tracking system according to claim 20, wherein the organism is an insect.

27. The tracking system according to claim 26, wherein at least one insect is marked with unique barcode DNA after ingestion of the crosslinked porous protein crystal comprising the synthetic library.

28. The tracking system according to claim 27, wherein the crosslinked porous protein crystal comprising the synthetic library is ingested by the at least one insect when the insect is a larva, pupa, adult, or any combination thereof.

29. The tracking system according to claim 20, wherein the synthetic library is recovered from the crosslinked porous protein crystal with less than 10% degradation.

30. The tracking system according to claim 20, wherein the synthetic library recovered from the crosslinked porous protein crystal is subjected to PCR, qPCR, ddPCR, rtPCR, next-generation sequencing, or any combination thereof to determine the unique barcode DNA for the organism.

31. The tracking system according to claim 20, wherein the synthetic library comprises about 10 million to about 200 million reads of the unique barcode DNA.

32. A method of marking an organism, said method comprising the steps of:
providing a tracking system comprising i) a synthetic library encoded with unique barcode DNA sequences and ii) a crosslinked porous protein crystal for ingestion by the organism, wherein the synthetic library is stored in the crosslinked porous protein crystal, and
marking the organism with at least one unique barcode DNA via ingestion of the crosslinked porous protein crystal by the organism.

33. The method of claim 32, wherein the synthetic library comprises at least one modular barcode library.

34. The method of claim 32, wherein the organism is an insect.

35. The method of claim 34, wherein the insect is a larva, pupa, adult, or any combination thereof.

36. The method of claim 34, wherein the insect is a mosquito.

37. The method of claim 32 further comprising a step of recovering at least one unique barcode DNA from the organism.

38. The method of claim 37, wherein the unique barcode DNA is subjected to PCR, qPCR, ddPCR, rtPCR, next-generation sequencing, or any combination thereof.

39. The method of claim 37, wherein the unique barcode DNA comprises about 5 base pairs (bp) to about 300 bp.


SEQUENCE LISTING (11 sequences)

SEQ ID NO: 1 - DNA, 124 bp, Artificial Sequence
  synthetic; FAM labeled 125mer
aggcgactcgacggtcttacgcgttacgtatgatatgcatcaccaccatcaccaataaccaacacctaaatttaacatccgagaattatggagcacgctagcgtacgctacggtcctaacgcgc

SEQ ID NO: 2 - DNA, 30 bp, Artificial Sequence
  synthetic; FWD primer for FAM labeled 125mer
taggcgactcgacggtcttacgcgttacgt

SEQ ID NO: 3 - DNA, 30 bp, Artificial Sequence
  synthetic; REV primer for FAM labeled 125mer
gcgcgttaggaccgtagcgtacgctagcgt

SEQ ID NO: 4 - DNA, 51 bp, Artificial Sequence
  synthetic; FWD primer for overhang PCR
acactctttccctacacgacgctcttccgatctccagtcctcaacaagctg

SEQ ID NO: 5 - DNA, 18 bp, Artificial Sequence
  synthetic; REV primer for overhang PCR
gttgaagccggttaccac

SEQ ID NO: 6 - DNA, 33 bp, Artificial Sequence
  synthetic; FWD primer for additional round 1 of overhang PCR
acactctttccctacacgacgctcttccgatct

SEQ ID NO: 7 - DNA, 44 bp, Artificial Sequence
  synthetic; REV primer for additional round 1 of overhang PCR
  misc_feature (19)..(26): n is a, c, g, t or u
ttctgggttcctcatcgcnnnnnnnngttgaagccggttaccac

SEQ ID NO: 8 - DNA, 33 bp, Artificial Sequence; identical to SEQ ID NO: 6
  synthetic; FWD primer for additional round 2 of overhang PCR

SEQ ID NO: 9 - DNA, 52 bp, Artificial Sequence
  synthetic; REV primer for additional round 2 of overhang PCR
gtgactggagttcagacgtgtgctcttccgatctttctgggttcctcatcgc

SEQ ID NO: 10 - DNA, 56 bp, Artificial Sequence
  synthetic; FWD primer for additional round 3 of overhang PCR
aatgatacggcgaccaccgagatctacactctttccctacacgacgctcttccgat

SEQ ID NO: 11 - DNA, 76 bp, Artificial Sequence
  synthetic; REV primer for additional round 3 of overhang PCR
  misc_feature (25)..(34): n is a, c, g, t or u
caagcagaagacggcatacgagatnnnnnnnnnnatattcacgtgactggagttcagacgtgtgctcttccgatct